SEQUENCE LISTING (39 sequences)

SEQ ID NO: 1 - protein, 1102 aa, unknown
  Arabidopsis CHR12 protein
MVAQQLQERCGGTSQEDPVETTKSLICALNYISRDLPLPPHLFTAVSSIYHGASSSSLSDSDVSPPLPTSPPANKAPYGADLMGEFEDALLKQRPDCESGSRLIQLLDNRNKSHIQRRLSELEELPSTRGEDLQAKCLLELYGLKLRELQGKVRTAVSSEFWLRLNCADVSSQVFDWGMMRLPRPFYGVGDPFAMEADDQFRKKRDAERLSRLEEEEKNLIETAKRKFFAEVLNAVREFQLQIQATQKRRRQRNDGVQAWHGRQRQRATRAEKLRLMALKSDDQEAYMKLVKESKNERLTTLLEETNKLLANLGAAVQRQKDAKLPEGIDLLKDSESDLSELDAPRSEPLQDLLPDQDIDITESDNNDDSNDLLEGQRQYNSAIHSIQEKVTEQPSLLEGGELRSYQLEGLQWMVSLFNNNLNGILADEMGLGKTIQTISLIAYLLENKGVPGPYLIVAPKAVLPNWVNEFATWVPSIAAFLYDGRLEERKAIREKIAGEGKFNVLITHYDLIMRDKAFLKKIEWYYMIVDEGHRLKNHESALAKTLLTGYRIKRRLLLTGTPIQNSLQELWSLLNFLLPHIFNSVQNFEEWFNAPFADRGNVSLTDEEELLIIHRLHHVIRPFILRRKKDEVEKFLPGKTQVILKCDMSAWQKVYYKQVTDMGRVGLQTGSGKSKSLQNLTMQLRKCCNHPYLFVGGDYNMWKKPEIVRASGKFELLDRLLPKLRKAGHRILLFSQMTRLIDVLEIYLTLNDYKYLRLDGTTKTDQRGLLLKQFNEPDSPYFMFLLSTRAGGLGLNLQTADTVIIFDSDWNPQMDQQAEDRAHRIGQKKEVRVFVLVSVGSVEEVILERAKQKMGIDAKVIQAGLFNTTSTAQDRREMLEEIMRKGTSSLGTDVPSEREINRLAARSEDEFWMFERMDEERRRKENYRARLMQEQEVPEWAYTTQTQEEKLNNGKFHFGSVTGKRKRKEIVYSDTLSELQWLKAVESGEDLSKLSMRYNRREENASNTKTSTSKKVIESIQTVSDGTSEEDEEEQEEERAKEMSGKQRVDKSEEEEEEGEEENDGKAIFKWNTHKKKRSRYSFTCSSSDSRAQSSNGSRRK

SEQ ID NO: 2 - DNA, 3460 bp, unknown
  Arabidopsis thaliana CHR12 cDNA
atggtggctcagcagctccaagagagatgcggcggcacttctcaagaagatccagttgagaccaccaagtctctgatctgtgctctcaattacatttctcgtgaccttcctttgcctcctcacctcttcaccgccgtttcttcaatctaccatggcgcttcttcttcttctctctctgactccgatgtctctccgccgctacctacttcgccgcctgcgaataaagctccctatggagctgatttgatgggtgaattcgaggatgcacttttgaagcagagaccagattgtgaatcaggctctcgattgatacaattactggataatcggaataagagtcatatccaacgccgcttatctgaacttgaagaattaccttctactagaggtgaagatttgcaggcaaagtgcttacttgagctttatgggcttaagctacgggaattgcaaggcaaggttcgaaccgcggtgagctcggaattctggctacgtctgaactgtgcagatgtcagtagccaagtatttgactggggcatgatgcggttgcctcgtccattttatggggttggagatccttttgctatggaagctgatgatcagttcagaaagaagcgtgacgctgagaggttatcacgtttagaagaagaggagaagaatctgattgaaactgccaagaggaaattctttgcagaagtactcaatgcagtacgtgaattccagttgcaaattcaggcgactcaaaaacgccgtaggcaaaggaatgatggtgtgcaggcttggcatgggagacaaagacaacgcgctacccgtgctgaaaagttgaggctcatggcccttaagtccgatgatcaagaggcatacatgaaactagtaaaggagagtaaaaatgaaagactaaccactcttttagaggagacaaacaaactccttgctaatttgggagctgctgttcaacgtcagaaagatgctaaacttccggaaggcattgatctacttaaagactcagaatctgacttgtctgaacttgatgctccaagaagtgaaccattgcaggatctgcttcctgatcaggatatagatattactgagtctgacaataacgatgactctaatgatttactagaaggacagaggcagtataactcggctatccattcaatacaagagaaggtgacggagcagccctcgcttctagaaggtggggaactacgatcctatcaattagaagggcttcaatggatggtttctctattcaacaataacctcaatggaatattagctgatgagatgggtttgggaaaaactattcaaacaatttctctgattgcgtatcttctggagaataaaggtgtgcctgggccttatcttatagttgctccaaaagctgtgctaccaaattgggtaaacgagtttgcaacatgggtaccaagcattgcagcttttctgtatgatggacgtttagaggaaaggaaagcaataagggaaaaaattgcaggagaaggaaaatttaatgtgttgataacccattatgatcttatcatgagagataaagcatttttaaagaaaattgagtggtactacatgattgttgatgaaggacatcggctgaagaaccacgaatctgctctagcaaagactttattaacaggctatcgtataaaacgcagacttctgttgaccgggacacccatacagaatagccttcaagaactatggtcattgcttaattttctccttcctcacatctttaactcggttcagaacttcgaggaatggtttaatgctcctttcgctgaccgtggtaatgtcagtctcacggatgaagaggagctcttgattattcaccgcctgcatcatgttataaggccgtttatactgagaaggaaaaaggacgaagtagagaaattccttcctggaaagacacaggtcatattgaagtgtgatatgtcggcgtggcagaaagtgtattacaaacaagttacagacatgggcagggttggccttcaaacaggatctgggaaatcaaagagtctgcaaaatctaacgatgcaattaagaaagtgttgtaaccatccttatctatttgttggaggagactacaatatgtggaagaaacctgagattgtgagagcttcagggaaatttgaactccttgatcgtctccttccaaaactgcgcaaggctggacataggattcttctcttctcccaaatgacccgtctcattgacgttcttgagatatacttgacacttaatgattacaaataccttagactcgatggtaccacaaagacagatcaaagagggcttttactgaagcaattcaacgagccagattctccctactttatgtttcttttgagcacgcgtgctggaggtctcggtttgaacttgcaaactgcagacactgttattatctttgacagtgattggaacccacaaatggaccaacaggccgaggatcgagctcatcgcatagggcagaagaaggaagtcagagtgtttgttttggttagcgttggctctgttgaagaagtaatattggagcgtgcaaagcaaaagatgggcattgacgccaaagtcatacaagctggacttttcaacacaacttccactgcacaagacagaagagagatgcttgaagagattatgcgtaaaggaacaagctcgctcgggacagatgtccccagtgagagagaaataaaccggctagcggctcgtagtgaggatgaattttggatgtttgagcgaatggatgaggagagacgaaggaaggagaattacagagcccgtctgatgcaagagcaagaagtccctgagtgggcgtacactactcagacccaagaggagaagttgaacaacggaaagttccattttggtagtgtcacaggtaagcgaaaacgtaaagagattgtttacagcgacacactaagcgagttacagtggctgaaagccgtggagagcggtgaagacctgtcaaaactctcaatgagatataataggagagaggagaacgcaagcaacacaaagacatcaacgagcaaaaaagtgatagaatctatccaaactgtgagtgatgggacgagcgaggaggatgaagaagagcaagaagaagaaagagcgaaggagatgagtggaaaacagagagtggacaagtctgaagaggaagaagaagaaggtgaagaagagaatgatggaaaggcaatattcaagtggaatactcataagaagaaaaggtctagatactcttttacgtgttcttcgtctgactctagagctcaaagttccaatggaagtagacgaaaatgaagaaggtgaggtgagatctgagtttgactttggtctcttcttctacgtgtttgtgtgtcttcttgccaatgtaacttatcccaatctaacatgtaatagtaatacatgtattttaattggtctctggttaagtttagctttttgttgttgt

SEQ ID NO: 3 - DNA, 3309 bp, unknown
  Arabidopsis thaliana CHR12 open reading frame (ORF)
atggtggctcagcagctccaagagagatgcggcggcacttctcaagaagatccagttgagaccaccaagtctctgatctgtgctctcaattacatttctcgtgaccttcctttgcctcctcacctcttcaccgccgtttcttcaatctaccatggcgcttcttcttcttctctctctgactccgatgtctctccgccgctacctacttcgccgcctgcgaataaagctccctatggagctgatttgatgggtgaattcgaggatgcacttttgaagcagagaccagattgtgaatcaggctctcgattgatacaattactggataatcggaataagagtcatatccaacgccgcttatctgaacttgaagaattaccttctactagaggtgaagatttgcaggcaaagtgcttacttgagctttatgggcttaagctacgggaattgcaaggcaaggttcgaaccgcggtgagctcggaattctggctacgtctgaactgtgcagatgtcagtagccaagtatttgactggggcatgatgcggttgcctcgtccattttatggggttggagatccttttgctatggaagctgatgatcagttcagaaagaagcgtgacgctgagaggttatcacgtttagaagaagaggagaagaatctgattgaaactgccaagaggaaattctttgcagaagtactcaatgcagtacgtgaattccagttgcaaattcaggcgactcaaaaacgccgtaggcaaaggaatgatggtgtgcaggcttggcatgggagacaaagacaacgcgctacccgtgctgaaaagttgaggctcatggcccttaagtccgatgatcaagaggcatacatgaaactagtaaaggagagtaaaaatgaaagactaaccactcttttagaggagacaaacaaactccttgctaatttgggagctgctgttcaacgtcagaaagatgctaaacttccggaaggcattgatctacttaaagactcagaatctgacttgtctgaacttgatgctccaagaagtgaaccattgcaggatctgcttcctgatcaggatatagatattactgagtctgacaataacgatgactctaatgatttactagaaggacagaggcagtataactcggctatccattcaatacaagagaaggtgacggagcagccctcgcttctagaaggtggggaactacgatcctatcaattagaagggcttcaatggatggtttctctattcaacaataacctcaatggaatattagctgatgagatgggtttgggaaaaactattcaaacaatttctctgattgcgtatcttctggagaataaaggtgtgcctgggccttatcttatagttgctccaaaagctgtgctaccaaattgggtaaacgagtttgcaacatgggtaccaagcattgcagcttttctgtatgatggacgtttagaggaaaggaaagcaataagggaaaaaattgcaggagaaggaaaatttaatgtgttgataacccattatgatcttatcatgagagataaagcatttttaaagaaaattgagtggtactacatgattgttgatgaaggacatcggctgaagaaccacgaatctgctctagcaaagactttattaacaggctatcgtataaaacgcagacttctgttgaccgggacacccatacagaatagccttcaagaactatggtcattgcttaattttctccttcctcacatctttaactcggttcagaacttcgaggaatggtttaatgctcctttcgctgaccgtggtaatgtcagtctcacggatgaagaggagctcttgattattcaccgcctgcatcatgttataaggccgtttatactgagaaggaaaaaggacgaagtagagaaattccttcctggaaagacacaggtcatattgaagtgtgatatgtcggcgtggcagaaagtgtattacaaacaagttacagacatgggcagggttggccttcaaacaggatctgggaaatcaaagagtctgcaaaatctaacgatgcaattaagaaagtgttgtaaccatccttatctatttgttggaggagactacaatatgtggaagaaacctgagattgtgagagcttcagggaaatttgaactccttgatcgtctccttccaaaactgcgcaaggctggacataggattcttctcttctcccaaatgacccgtctcattgacgttcttgagatatacttgacacttaatgattacaaataccttagactcgatggtaccacaaagacagatcaaagagggcttttactgaagcaattcaacgagccagattctccctactttatgtttcttttgagcacgcgtgctggaggtctcggtttgaacttgcaaactgcagacactgttattatctttgacagtgattggaacccacaaatggaccaacaggccgaggatcgagctcatcgcatagggcagaagaaggaagtcagagtgtttgttttggttagcgttggctctgttgaagaagtaatattggagcgtgcaaagcaaaagatgggcattgacgccaaagtcatacaagctggacttttcaacacaacttccactgcacaagacagaagagagatgcttgaagagattatgcgtaaaggaacaagctcgctcgggacagatgtccccagtgagagagaaataaaccggctagcggctcgtagtgaggatgaattttggatgtttgagcgaatggatgaggagagacgaaggaaggagaattacagagcccgtctgatgcaagagcaagaagtccctgagtgggcgtacactactcagacccaagaggagaagttgaacaacggaaagttccattttggtagtgtcacaggtaagcgaaaacgtaaagagattgtttacagcgacacactaagcgagttacagtggctgaaagccgtggagagcggtgaagacctgtcaaaactctcaatgagatataataggagagaggagaacgcaagcaacacaaagacatcaacgagcaaaaaagtgatagaatctatccaaactgtgagtgatgggacgagcgaggaggatgaagaagagcaagaagaagaaagagcgaaggagatgagtggaaaacagagagtggacaagtctgaagaggaagaagaagaaggtgaagaagagaatgatggaaaggcaatattcaagtggaatactcataagaagaaaaggtctagatactcttttacgtgttcttcgtctgactctagagctcaaagttccaatggaagtagacgaaaatga

SEQ ID NO: 4 - DNA, 4850 bp, unknown
  Arabidopsis thaliana genomic CHR12 DNA from ecotype WS
atggtggctcagcagctccaagagagatgcggcggcacttctcaagaagatccagttgagaccaccaagtctctgatctgtgctctcaattacatttctcgtgaccttcctttgcctcctcacctcttcaccgccgtttcttcaatctaccatggcgcttcttcttcttctctctctgactccgatgtctctccgccgctacctacttcgccgcctgtaattcactctcgagcccttctccagcctcaccctagattttgatgtttgcccctttttgtgttttgtgctttgtttgggaggttttgaatcgatttatgtggtcctgtgatttgtttttgtagctttgctttcgagtttatgttcaaagttttctcctttttcgagtttctatggaaattagatatcttcagtggcttgttttgtcgaattacagtctaaaggtttttgctttaatctgtggtttggtctctttgttgtttttgcaggcgaataaagctccctatggagctgatttgatgggtgaattcgaggatgcacttttgaagcagagaccagattgtgaatcaggctctcgattgatacaattactggataatcggaataagagtcatatccaacgccgcttatctgaacttgaaggttattatcgtgtttattactcctaagttgcacttcaatccattctcaagttttgattttgttatgcttggcaacagtttttttgttactggagaactattgtcaattatgttactaaaaccctttattcttcaagggggatgctttgtgcttactctttgtgtttttgaatcagaattaccttctactagaggtgaagatttgcaggcaaagtgcttacttgagctttatgggcttaaggtatcatcgttgctagatgttctcattgatcgttgttgttatgttacattgtttgaattgttatatctttgacattgacactttacctggtgagttacagctacgggaattgcaaggcaaggttcgaaccgcggtgagctcggaattctggctacgtctgaactgtgcagatgtcagtagccaagtatttgactggggcatgatgcggttgcctcgtccattttatggggttggagatccttttgctatggaagctgatgatcagttcagaaagaagcgtgacgctgaggtgatttaccttttattcagctagcatattctggtgcaacttttttagttcctggattttattttaacctatgataatgaagtttcctgatgagataaggccccttttgaactatggctcatatatcttagcagtacaactagacatgtgtataaatatatcactagattctgctttgttggaactcatttagatggatttaatagaattgaaatgaaaagtgtacacaatttctagttgttatgacatgattttattttatagctttcaatcttcgtgattggcattgctgatgtgttaaaagtctttgttcagaggttatcacgtttagaagaagaggagaagaatctgattgaaactgccaagaggaaattctttgcagaagtactcaatgcagtacgtgaattccagttgcaaattcaggcgactcaaaaacgccgtaggcaaaggaatgatggtgtgcaggtatgatagagtttacttacgcggttattgtgatagtagttccagttatcttttaatattaccaatcaatatacaaacatctaaaatttattttgtgtgtacattattcgaacaggcttggcatgggagacaaagacaacgcgctacccgtgctgaaaagttgaggctcatggcccttaagtccgatgatcaagaggcatacatgaaactagtaaaggagagtaaaaatgaaagactaaccactcttttagaggagacaaacaaactccttgctaatttgggagctgctgttcaacgtcagaaagatgctaaacttccggaaggcattgatctacttaaagactcagaatctgacttgtctgaacttgatgctccaagaagtgaaccattgcaggatctgcttcctgatcaggatatagatattactgagtctgacaataacgatgactctaatgatttactagaaggacagaggcagtataactcggctatccattcaatacaagagaaggtaacgttgatctctatatacttagtagctgtatggtgaactgtttaaaccactgccatctcagagaccagggaatttaaaatagcaatatatacttttaacaggtgacggagcagccctcgcttctagaaggtggggaactacgatcctatcaattagaagggcttcaatggatggtttctctattcaacaataacctcaatggaatattagctgatgagatgggtttgggaaaaactattcaaacaatttctctgattgcgtatcttctggagaataaaggtgtgcctgggccttatcttatagttgctccaaaagctgtgctaccaaattgggtaaacgagtttgcaacatgggtaccaaggtatgttgcaaacaatatttctcttggtactgagaaattgcactggtatgaatgttatgtctgaaaatcagcctttttgtttttttgacagcattgcagcttttctgtatgatggacgtttagaggaaaggaaagcaataagggaaaaaattgcaggagaaggaaaatttaatgtgttgataacccattatgatcttatcatgagagataaagcatttttaaagaaaattgagtggtactacatgattgttgatgaaggacatcggctgaagaaccacgaatctgctctagcaaagactttattaacagggtacatctctatattcatctgtctgaagtgaatgatgttggctaactattatgtttctaacaaaaatttatccgggatgcagctatcgtataaaacgcagacttctgttgaccgggacacccatacagaatagccttcaagaactatggtcattgcttaattttctccttcctcacatctttaactcggttcagaacttcgaggaatggtttaatgctcctttcgctgaccgtggtaatgtcagtctcacggatgaagaggagctcttgattattcaccgcctgcatcatgtgggttctctagctgtctttccttaaatttcttatcaaaatttgtatgcactgttttaatgtaattcactctgtcactttcaggttataaggccgtttatactgagaaggaaaaaggacgaagtagagaaattccttcctggaaagacacaggtcatattgaagtgtgatatgtcggcgtggcagaaagtgtattacaaacaagttacagacatgggcagggttggccttcaaacaggtttgtagcttaactcacgtatactctctccctaatgttgcactcatctttgaggtaagagaaaatgaatcagaaagaggagctgattttatgcaaacgtaatcaccatgtgcaggatctgggaaatcaaagagtctgcaaaatctaacgatgcaattaagaaagtgttgtaaccatccttatctatttgttggaggagactacaatatgtggaagaaacctgagattgtgagagcttcagggaaatttgaactccttgatcgtctccttccaaaactgcgcaaggctggacataggattcttctcttctcccaaatgacccgtctcattgacgttcttgagatatacttgacacttaatgattacaaataccttagactcgatggtaccacaaagacagatcaaagagggcttttactgaagcaattcaacgagccagattctccctactttatgtttcttttgagcacgcgtgctggaggtctcggtttgaacttgcaaactgcagacactgttattatctttgacagtgattggaacccacaaatggaccaacaggccgaggatcgagctcatcgcatagggcagaagaaggaagtcagagtgtttgttttggttagcgttggctctgttgaagaagtaatattggagcgtgcaaagcaaaagatgggcattgacgccaaagtcatacaagctggacttttcaacacaacttccactggtatgtctgtccgaaagaacaatatgagggcttctgctttctatatcgatatggtaatctcttgatgtggtttttgtgattatatcaacagcacaagacagaagagagatgcttgaagagattatgcgtaaaggaacaagctcgctcgggacagatgtccccagtgagagagaaataaaccggctagcggctcgtagtgaggatgaattttggatgtttgagcgaatggatgaggagagacgaaggaaggagaattacagagcccgtctgatgcaagagcaagaagtccctgagtgggcgtacactactcagacccaagaggagaagttgaacaacggaaagttccattttggtagtgtcacaggtaagcgaaaacgtaaagagattgtttacagcgacacactaagcgagttacagtggctgaaagccgtggagagcggtgaagacctgtcaaaactctcaatgagatataataggagagaggagaacgcaagcaacacaaagacatcaacgagcaaaaaagtgatagaatctatccaaactgtgagtgatgggacgagcgaggaggatgaagaagagcaagaagaagaaagagcgaaggagatgagtggaaaacagagagtggacaagtctgaagaggaagaagaagaaggtgaagaagagaatgatggaaaggcaatattcaagtggaatactcataagaagaaaaggtctagatactcttttacgtgttcttcgtctgactctagagctcaaagttccaatggaagtagacgaaaatga

SEQ ID NO: 5 - DNA, 1480 bp, unknown
  Promoter of Arabidopsis AtCHR12 gene
gttagtggagcctttatgagccactccttaaacttttaaatcgagcttatatattataagcaatgcttacacatgttaaggaaccattggttaacatagttcaaaaaatttactagacaaagaataaaaataaagaattttatgtattggccacatagagttattgtttgtctcccattgcttttgttgacgcatgacatccacatgaaaatatcattctcatccaaaactgttgtctcactcacatatcatccaagcaaactataactttcacaaaggagatgtcaatttacatgaaaaaggcttttatttaaggtgaagtgcggataagaatagtgattcaaacgaacaaacacagtcatggatcgatctattccattactagaacaaggatctttaattataacccattcaatctaaagttattattagccaacacagtagtcaaaaaatagggaagataaagcaacattcagtttcctatacacacaaaattttactagataacaagagtcgatcgacaataacatggacaaacgtacatctacaagtgggacaatgaacgatttgaatcatacgtacatatacacccattgtaattcacgactgaaagaaatgggacaaagaatctctaggcaacactatctcccaacagcaaacaaattactatttccgagaagaacaaaaaatgtgactcacactcacttaaggttttgaaattacaagattttagaaattggaatggacatgtatatgtattgtcggtgtgttgtcttataacctcttaatgggaatcggattcaaaacaaaccctctcattccacgtttgtcaagcacgtgcttcttcaagaatttgaaattttggtttaacggtacactcgatttgatcagaacgagacaattaattacaattaaatacaaatttagttactgtcggtagttttttttttccgaatgcttcctgtcgcatatattgttttggttctatgaactcttattagcatataccagaatctgttgaaccatacaaatttactatattttctcggtgactctctatatagcagcaaaccattagcgagactagattatgaaccttctaaacattaactaaaatactcagtttcaagcccaatcaatatcaatatatagcccgcagatgccgatcaaatgggcctgtccaacggcccattaataaaacgggggttaaaaatgtaaatacaagaagatagatgtttggtgagtcctaaaaatcgtaaaccagatttccaaatttctctccgtctcatctttgtctcggtctctctctcgagtgtgtgtgtgtctaaaggcaaagagagagatttgagaaaactgaattacaaaaaaaaaaaaaaaagacaacgaaaaaaaagacatcaaatttgcgaaagttgaaatcgaaaacggtgaatacaagttcccgccatggtggctcagcagctccaagagagatgcggcg

SEQ ID NO: 6 - DNA, 19 bp, artificial
  primer
ccaaagtgacatctcatgg

SEQ ID NO: 7 - DNA, 21 bp, artificial
  primer
cttaccttttttcttgtagtg

SEQ ID NO: 8 - DNA, 21 bp, artificial
  primer
gcctcaccctagattttgatg

SEQ ID NO: 9 - DNA, 22 bp, artificial
  primer
gcgtggaccgcttgctgcaact

SEQ ID NO: 10 - DNA, 19 bp, artificial
  primer
gcaaacggagcagtttggc

SEQ ID NO: 11 - DNA, 21 bp, artificial
  primer
gcaatagagtacaaaccaatc

SEQ ID NO: 12 - DNA, 20 bp, artificial
  primer
gatgcagctcaaagtccctg

SEQ ID NO: 13 - DNA, 21 bp, artificial
  primer
gacaccattaccatcaagaac

SEQ ID NO: 14 - DNA, 18 bp, artificial
  primer
gcgtcaccaaagtcaccc

SEQ ID NO: 15 - DNA, 21 bp, artificial
  primer
gttcaacacatctgatcaaag

SEQ ID NO: 16 - DNA, 20 bp, artificial
  primer
gaggaatgggcacaaacctc

SEQ ID NO: 17 - DNA, 18 bp, artificial
  primer
gaagacgcatcgccaccg

SEQ ID NO: 18 - DNA, 20 bp, artificial
  primer
gaggaatgggcacaaacctc

SEQ ID NO: 19 - DNA, 18 bp, artificial
  primer
gaagacgcatcgccaccg

SEQ ID NO: 20 - DNA, 20 bp, artificial
  primer
gcgtgagctagctcaagcag

SEQ ID NO: 21 - DNA, 20 bp, artificial
  primer
caggaggtgatatggctatg

SEQ ID NO: 22 - DNA, 21 bp, artificial
  primer
cctccacaatccatgtccttc

SEQ ID NO: 23 - DNA, 16 bp, artificial
  primer
cgttctctgcttcggc

SEQ ID NO: 24 - DNA, 19 bp, artificial
  primer
cgttgtaccgcatttgaag

SEQ ID NO: 25 - DNA, 20 bp, artificial
  primer
ctccaattctcttggtgctc

SEQ ID NO: 26 - DNA, 20 bp, artificial
  primer
cactctctctgatgttaacg

SEQ ID NO: 27 - DNA, 18 bp, artificial
  primer
ccagcatgtttctccggc

SEQ ID NO: 28 - DNA, 18 bp, artificial
  primer
cttacgagacgccggagg

SEQ ID NO: 29 - DNA, 22 bp, artificial
  primer
catacagcaaaactcgttggac

SEQ ID NO: 30 - DNA, 20 bp, artificial
  primer ubiquitin
atgcagayttttgtgaagac

SEQ ID NO: 31 - DNA, 18 bp, artificial
  primer ubiquitin
accaccacgragacggag

SEQ ID NO: 32 - DNA, 32 bp, artificial
  primer
ggatcctcatgaaggctcagcagctccaagag

SEQ ID NO: 33 - DNA, 23 bp, artificial
  primer
ccttctaattgataggatcgtag

SEQ ID NO: 34 - DNA, 22 bp, artificial
  primer
ggctatccattcaatacaagag

SEQ ID NO: 35 - DNA, 20 bp, artificial
  primer
gggttccaatcactgtcaag

SEQ ID NO: 36 - DNA, 22 bp, artificial
  primer
caattcaacgagccagattctc

SEQ ID NO: 37 - DNA, 26 bp, artificial
  primer
ctcgagtcattttcgtctacttccat

SEQ ID NO: 38 - DNA, 23 bp, artificial
  primer
gttagtggaagcctttatgagcc

SEQ ID NO: 39 - DNA, 20 bp, artificial
  primer
gccaccatggcgggaacttg

(19) United States
(12) Patent Application Publication
(10) Pub. No.: US 2010/0138962 A1
(43) Pub. Date: Jun. 3, 2010

(54) USE OF PLANT CHROMATIN REMODELING GENES FOR MODULATING PLANT ARCHITECTURE AND GROWTH

(75) Inventors: Ludmila Mlynárová, Wageningen (NL); Ton Bisseling, Ede (NL)

Correspondence Address: FOLEY AND LARDNER LLP;SUITE 500, 3000 K STREET NW, WASHINGTON, DC, 20007, US

(73) Assignee: Wageningen Universiteit

(21) Appl. No.: 12/305,881

(22) PCT Filed: Jun. 20, 2007
(86) PCT No.: PCT/NL2007/050297

§ 371 Date: Jul. 1, 2009

(30) Foreign Application Priority Data
Jun. 20, 2006 (NL) PCT/NL06/50141

(51) Int. Cl. A01H 5/00; C12N 15/29; C12N 15/82; C12Q 1/68

(52) U.S. Cl. 800/290; 800/298; 800/320.1; 800/320.2; 800/320.3; 800/317.4; 800/317; 800/320; 800/306; 800/312; 800/313; 800/314; 800/305; 800/318; 800/322; 800/317.2; 536/23.6; 435/320.1; 800/278; 435/6

(57) ABSTRACT

The present invention relates to the field of transgenic plants having modified growth and to the use of chromatin remodeling genes, in particular Arabidopsis gene (AtCHR12) and homologs and orthologs thereof, for making such plants. AtCHR12 is involved in the flexible modulation of growth characteristics in development, notably after the perception of environmental stress. It demonstrates the intimate relationships between environment, chromatin and growth in plants.

### FIELD OF THE INVENTION

The present invention relates to the field of transgenic plants wherein chromatin remodeling genes, in particular AtCHR12 genes and/or homologous or orthologs thereof, are (over) expressed or downregulated using e.g. RNA interference. The transgenic plants or parts thereof have modified growth characteristics, such as a prolonged or more severe (reversible) dormancy-like growth arrest (or growth retardation) of plants or plant parts (e.g. dwarf or semi-dwarf plants); and/or delayed or suppressed bolting; and/or a higher or more uniform seed dormancy and better control of dormancy maintenance and breaking; and/or a less severe dormancy-like growth arrest (or growth retardation) of plants or plant parts and/or a prolonged life-span and/or altered dormancy characteristics, such as an extended dormancy period or a more uniform dormancy length. Especially the growth responses of plants exposed to biotic and/or abiotic stress conditions (such as arrest/retardation of growth of the stem or inflorescences) can be modulated using the present invention, essentially without altering the phenotype of the plants during non-stress conditions. Transgenic plants are provided which resume normal growth and development once the stress conditions have been removed again (i.e. the growth modification is reversible and stress dependent). Also provided are methods for making and selecting such plants, as well as methods for isolating other genes involved in dormancy-like growth arrest or growth retardation. Further, methods for identifying orthologs or homologues of AtCHR12 genes and/or natural or induced mutants of AtCHR12 genes are provided, as well as marker assisted selection methods for transferring such genes into crop plants or combining particular alleleles of such genes in crop plants Also non-transgenic crop plants comprising such AtCHR12 alleles are provided.

### BACKGROUND OF THE INVENTION

Various environmental stresses cause adverse effects on the growth of plants. To cope with abiotic stresses such as excessive heat, cold, flooding, drought or desiccation, plants adapt with a wide range of responses at the molecular, cellular and whole-plant level (Zhu, J. K., Hasegawa, P. M., and Bressan, R. A., 1997, Critical Reviews in Plant Sciences 16, 253-277). Several proteins are synthesized in plants in response to stress. These include proteins taking part in signal transduction, such as transcription factors, RNA-binding proteins and other (Shinozaki, K., and Yamaguchi-Shinozaki, K., 2000), Curr Opin Plant Biol 3, 217-223; Xiong, L., and Zhu, J. K., 2001, Physiol Plant 112, 152-166) and various proteins that counteract unfavorable conditions (Smallwood, M. F., Calvert, C. M., and Bowles, D. J., 1999, Plant responses to environmental stress. Oxford, UK: BIOS Scientific Publishers). As a result, plants possess finely orchestrated mechanisms to reversibly reduce their growth and/or metabolism in response to adverse stress.

One of the general responses of plants to potentially adverse environmental conditions is a partial or complete arrest of growth to adapt to the new environment. Slower or stopped growth is considered to be an adaptive feature for survival allowing plants to employ multiple resources to combat stress (Zhu, J. K., 2001, Trends Plant Sci 6, 66-71). In such growth arrest, there is generally little or no decrease in the structural or functional integrity of the cell and tissues (Storey, K. B., 2001, Molecular mechanisms of metabolic arrest: life in limbo. Oxford, UK: BIOS Scientific Publishers). Generally, growth resumes immediately after the environmental limitations are overcome (Rohde, A., Van Montagu, M. and Boerjan, W., 1999, Plant Cell and Environment 22, 261-270).

Expression of numerous genes has to be modulated to achieve appropriate plant responses to stress (Arnholdt-Schmitt, B., 2004, Plant Physiol 136, 2579-2586). Necessary changes in expression patterns are thought to require the alteration of chromatin structure at promoters and other regulatory DNA regions mediated by chromatin remodeling enzymes (Aalfs, J. D., and Kingston, R. E., 2000, Trends Biochem Sci 25, 548-555). Such enzymes modulate the chromatin state into either an “open” (activation of transcription) (Narlikar et al., 2002, Cell 108, 475-487) or a “closed” (repression of transcription) configuration (Harikrishnan, et al. 2005, Nat Genet 37, 254-264).

The importance of chromatin remodeling in the transcriptional response to stress was described in yeast (Damelin, M et al. 2002, Mol Cell 9, 563-573; Mizuno, K. et al., 2001, Genetics 159, 1467-1478) and mammalian cells (de La Serna, I. L. et al. 2000, Mol Cell Biol 20, 2839-2851). In plants, chromatin remodeling proteins were demonstrated to take part in regulation of flowering time and vemalizaton (Noh, Y. S. and Amasino, R. M., 2003, Plant Cell 15, 1671-1682; Gendall, A. R., Levy, Y. Y., Wilson, A. and Dean, C., 2001, Cell 107, 525-535). Recently, chromatin remodelling proteins were implicated in both acclimation and adaptation in response to UV-B in maize (Casati, P., Stapleton, A. E., Blum, J. E. and Walbot, V., 2006, Plant J 46, 613-627).

Prominent remodelers of chromatin are the ATPase-dependent remodeling complexes (remodelers). These large multisubunit complexes use ATP hydrolysis locally disrupt or alter the topology of DNA (Tsukiyama, T. 2002, Nat Rev Mol Cell Biol 3, 422-429). The protein composition of such remodeling complexes can be very dynamic (Olave, I. A. et al. 2002, Annu Rev Biochem 71, 755-781) and present a heterogeneous mix of protein subunits, that are assembled combinatorially. The particular composition of proteins in a remodeling complex is thought to be associated with the particular cellular function of that complex (Kadam, S., and Emerson, B. M., 2003, Mol Cell 11, 377-389). Recently it was demonstrated that for example actin and actin-related proteins are part of remodeling complexes (Olave, 2002, supra; Meagher, et al. 2005, Plant Physiol 139, 1576-1585). The proteins within such complexes can interact with the basal transcriptional machinery and/or with gene-specific DNA-binding factors (Peterson, C. L., and Workman, J. L., 2000, Curr Opin Genet Dev 10, 187-192). This way, remodeling complexes play an important role in the regulation of expression of the eukaryotic genes (Becker, P. B., and Horz, W., 2002, Annu Rev Biochem 71, 247-273; Fan, H. Y. et al. 2003, Mol Cell 11, 1311-1322), notably in development (Kennison, J. A., 1995, Annu Rev Genet 29, 289-303; Vignali, M., et al., 2000, Mol Cell Biol 20, 1899-1910).

Currently, four different classes of remodeling complexes are recognized based on the type of their ATPase subunit. These are known as SWI/SNF, ISWI, Mi-2 and Ino80 (Mohrmann, L., and Verrijzer, C. P., 2005, Biochim Biophys Acta 1681, 59-73). The yeast SWI/SNF family was the first chromatin remodeling complex described (Sudarsanam, P., and Winston, F., 2000, Trends Genet 16, 345-351). Its active components are highly conserved from yeast to humans. SWI/SNF-based complexes include the yeast SWI2/SNF2 and the related RSC complex, the Drosophila Brahma complex and the human BRM and BRG1 complexes (Tsukiyama, 2002, supra; Martens, J. A., and Winston, F., 2003, Curr Opin Genet Dev 13, 136-142). They all contain an ATPase subunit homologous to yeast SWI2 ATPase. The human remodeling complexes contain two ATPase subunits, hBRM and hBRG1, Drosophila contains only a single ATPase, Brahma (BRM) (Martens and Winston, 2003 supra). The typical feature of the SWI2/SNF2 class of ATPase subunits is the bromodomain. This domain recognizes acetylated lysines in histones (Hassan, A. H. et al., 2002, Cell 111, 369-379; Ladurner, A. G., et al. 2003, Mol Cell 11, 365-376; Marmorstein, R., and Berger, S. L. 2001, Gene 272, 1-9) and is supposed to target the complex to (hyper)acetylated chromatin, although removal of the bromodomain does not significantly affect the function of Brahma (Elfring, L. K., et al. 1998, Genetics 148, 251-265).

The Arabidopsis thaliana genome contains no less than 42 loci encoding putative SNF2-like ATPase subunits (see http://www.chromdb.org). Until now the function of ten of these loci have been characterized (Hsieh, T. F., and Fischer, R. L. 2005, Annu Rev Plant Biol 56, 327-351). All of these operate as modifiers of transcriptional or epigenetic regulation in plant development (Reyes, J. C., et al. 2002, Plant Physiol 130, 1090-1101; Wagner, D. 2003, Curr Opin Plant Biol 6, 20-28).

More distantly related members of SNF2 family, such as DDM1 (Jeddeloh, J. A., et al. 1998, Genes Dev 12, 1714-1725) and MOM1 (Amedeo, P., et al., 2000, Nature 405, 203-206), participate in epigenetic regulation. DRD1, a member of RAD54/ATRX family (Kanno, T. et al., 2004, Curr Biol 14, 801-805), is involved in the maintenance of RNA-directed non-CpG methylation. GYMNOS/PICKLE, that is encoding a protein of the CHD3 family, acts as repressor of embryonic programs after germination (Ogas, J. et al., 1997, Science 277, 91-94; Li, H. C., et al., 2005, Plant J 44, 1010-1022). Two ISWI-type genes have been characterized. PIE is involved in control of flowering time (Noh, Y. S., and Amasino, R. M., 2003, Plant Cell 15, 1671-1682) and CHR11 is essential for nuclear proliferation during female gametogenesis (Huanca-Mamani, W. et al., 2005, Proc Natl Acad Sci USA 102, 17231-17236). SWI3-type proteins affect embryogenesis as well as both vegetative and reproductive development (Zhou, C. et al. 2003, Plant Mol Biol 52, 1125-1134; Sarnowski, T. J. et al., 2002, Nucleic Acids Res 30, 3412-3421).

The subfamily most close to the SNF2/Brahma-type ATPase consists of four loci: SYD, AtBRM, AtCHR23 and AtCHR12 (Verbsky, M. L., and Richards, E. J. 2001, Curr Opin Plant Biol 4, 494-500). The first two have already been characterized in more detail. AtBRM is the Arabidopsis homolog that is most close to Drosophila Brahma (Farrona, S., et al., 2004, Development 131, 4965-4975). This is the only Arabidopsis Brahma-type ATPase containing the sequence related to the bromodomain. Silencing AtBRM by RNA interference demonstrated that this gene is required for proper vegetative and reproductive development. The silenced plants had reduced size, curled leaves, reduced inflorescence meristems, smaller petals, stamen and reduced fertility. AtBRM is strongly expressed in meristems, young organs, and in tissues with rapidly dividing cells (Farrona, 2004, supra). A loss-of-function mutation in SYD was identified in a screen for enhancers of a weak leafy (LFY) allele (Wagner, D., and Meyerowitz, E. M., 2002, Curr Biol 12, 85-94). The syd mutant displayed pleiotropic morphological phenotypes, such as short stature, slow growth, leaf polarity defects, ovule growth arrest and loss of maintenance of the shoot apical meristem. SYD was shown to function as a LFY-dependent repressor of the meristem identity switch in floral transition, most notably in the non-inductive photoperiod. Recently WUSCHEL (WUS) was identified as the first direct biologically important target of the SYD in the shoot apical meristem of Arabidopsis (Kwon, C. S., et al. 2005, Genes Dev 19, 992-1003). Both SYD and AtBRM act as repressors of the phase transition in non-inductive conditions, because their mutants flower earlier than wild-type plants. The similarity between these two mutants suggests some redundancy in the function of the genes.

So far no function of the Arabidopsis chromatin remodeling gene AtCHR12 has been found. The loss-of-function Arabidopsis mutant ecotype Columbia (carrying a T-DNA insertion in exon 1 of AtCHR12 (SALK_{—}105458) showed no visible phenotypic change compared to the wild type plant.

Surprisingly, the present inventors found an involvement of ATPase chromatin remodeling genes in stress responses in plants. This finding can be used to generate transgenic plants having a (temporarily) altered architecture and/or growth (both growth rate and growth period), which is especially prominent under stress conditions and disappears again when the stress conditions are removed. Such transgenic plants have, for example, a dwarfed or semi-dwarfed architecture under stress conditions compared to wild type plants, which improves both survival rate and/or yield (by minimizing yield loss under stress conditions).

The invention can also be used to delay or prevent bolting or flowering time/transition. Many vegetable crops become unusable if they bolt precociously. Preventing or delaying bolting in annual or biennial crops such as lettuce and potato plants means that they can be grown for longer periods (and have an extended harvest period) and give higher yields because plants do not need to invest resources in making flowers. In addition the time of planting or seeding can be adapted accordingly in bolting resistant lines or cultivars. Thus, a plant in which, for example, floral transition (bolting) is promoted by low temperature (e.g. vernalization) and/or long day lengths (LD) can be modified into a (recombinant) plant which is delayed in bolting or is resistant to bolting and which can therefore be grown under environmental conditions which otherwise would induce bolting and flower development. For example, such a plant could be grown in early spring or be planted in autumn, while it would otherwise only be suitable for planting/seeding in late spring or summer.

In addition, dormancy periods of vegetative tissues or organs can be modified, especially extended, and uniformity of the transition from a dormant to an active state can be increased. This is especially useful in root or tuber crops, such as potato, where the length of dormancy of the potato tubers after harvest varies greatly depending on cultivar and storage conditions (especially temperature and aeration). Thus, the time between harvest until dormancy-break may vary from 2 months to 5-6 months depending on cultivar and storage conditions, resulting in different time points of sprouting (dormancy-break). In one embodiment transgenic potato plants having an extended dormancy period and/or a more uniform length of the dormancy period are provided.

Also, seed dormancy which is a reflection of embryonic growth arrest can be modified using CHR12 genes according to the invention. For example, seed dormancy strength (% of seeds germinating), maintenance, breaking and cycling, of primary dormancy and/or secondary dormancy) can be controlled and modulated.

It is also an embodiment of the invention to identify wild type CHR12 alleles (e.g. homologues or orthologs of the Arabidopsis alleles), natural mutant alleles and/or to generate induced mutant CHR12 alleles and to use these alleles (using e.g. marker assisted selection methods) to generate non-transgenic plants and plant parts having altered architecture and/or growth phenotypes.

It is also noted that transgenic plants and plant parts described herein include cis-genesis, i.e. the generation of plants having genes introduced from the same plant species or gene-pool whereby the transgenic plant should be treated by regulatory authorities as non-GMO (see Jacobsen and Schouten, 2007, Trends in Biotechnology Vol 25: 219-223).

### GENERAL DEFINITIONS

“Root vegetables” or “root and tuber vegetables” or “root and tuber crops” is a generic term used herein to refer to plant storage organs growing underground, which are harvested and consumed by humans and animals. This term encompasses anatomically and developmentally different tissue types, such as “true roots” (e.g. turnip roots, carrot, sugar beet, etc.), “tuberous roots” (e.g. sweet potato, cassaya, etc.) and various modified underground stems. Modified stems can be subdivided into “corn” (e.g. taro), “Rhizomes” and “tubers” (e.g. potato, yam, etc.).

A “bulb” is an underground vertical shoot that has modified leaves (or thickened leaf bases) that are used as food storage organs. Bulbs include for example onions.

The term “geophyte” encompasses both (edible) underground bulbs and root vegetables as defined above.

“Growth arrest or retardation” refers herein to a temporary resting state in response to adverse environmental conditions of any plant structure. Growth arrest, also termed pre-dormancy (referred herein to as “dormancy-like”), is reversible in that if the plant is returned to favorable growing conditions it will resume growth. In contrast in true dormancy, which is under endogenous control, growth will not resume even if the plant is returned to optimal growing conditions. Therefore, “growth arrest” or “growth retardation” or “dormancy-like growth arrest or retardation” as used herein refers to a (statistically significant) reduction in the growth rate of one or more plant tissues or organs (such as inflorescences, internodes of the stem, apical or axillary buds, lateral or axillary shoots, leaves, fruit, seeds, tubers, roots, etc.) at one or more times during the life of the plant or under certain environmental conditions (e.g. under biotic and/or abiotic stress conditions). This includes varying degrees of growth arrest, ranging from arrest which is almost not visible to complete growth arrest. The degree of growth arrest is in certain embodiments “gene dosage dependent”, i.e. it correlates with the expression level of the transgene.

“Modified growth” refers to either a dormancy-like growth arrest or retardation as defined above (especially in plants expressing a functional ATCHR12 protein according to the invention) or to a relative reduction in dormancy-like growth arrest, i.e. leading to a relatively higher growth rate of one or more plant tissues or organs at one or more times during the life of the plant (especially in AtCHR12 knock-down or silenced plants) compared to a suitable control (e.g. non-transgenic control or empty-vector transformant). For example, under stress conditions the growth arrest or retardation of a transgenic plant wherein AtCHR12 is down-regulated is significantly reduced compared to the non-transgenic control under the same stress conditions. Thus, the transgenic stressed plant may have essentially the same growth phenotype as a non-stressed plant.

“Stress-induced” or “stress dependent” or “temporal” or “reversible” modified growth or dormancy-like growth arrest (or retardation) refers to the growth rate mainly being modified during exposure to one or more (biotic and/or abiotic) stress conditions, while growth resumes to a normal level upon elimination of the stress.

“Stress” refers to conditions or pressures of physical, chemical or biological origin acting on a plant or plant cells which may result in yield loss and/or quality loss of a plant, but which is not lethal to the plant.

“Non-stress conditions” refer herein to conditions under which physiology and development are normal or optimal.

“Biotic stress” refers to stress caused by biotic (live) agents, such as fungi, viruses, mycoplasma like organisms, insects, bacteria, nematodes etc. (i.e. especially plant pests and pathogens).

“Abiotic stress” refers to stress caused by abiotic (non-living) agents, such as temperature stress (cold/freezing, heat), salinity (salt), wind, metals, day-length (photoperiod), water-stress (such as too little or too much water availability, i.e. drought, dehydration, water-logging, etc.), wounding, radiation, nutrient availability (e.g. nitrogen or phosphor deficiency), etc.

The term “nucleic acid sequence” (or nucleic acid molecule) refers to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein” or “polypeptide” are used interchangeably and refer to molecules consisting of a chain of amino acids, without reference to a specific mode of action, size, 3 dimensional structure or origin. A “fragment” or “portion” of a ATCHR12 protein may thus still be referred to as a “protein”. An “isolated protein” is used to refer to a protein which is no longer in its natural environment, for example in vitro or in a recombinant bacterial or plant host cell.

The term “gene” means a DNA sequence comprising a region (transcribed region), which is transcribed into an RNA molecule (e.g. an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′non-translated sequence comprising e.g. transcription termination sites.

A “chimeric gene” (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

A “3′ UTR” or “3′ non-translated sequence” (also often referred to as 3′ untranslated region, or 3′end) refers to the nucleic acid sequence found downstream of the coding sequence of a gene, which comprises, for example, a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). An active protein in certain embodiments refers to a protein having a dominant-negative function due to a repressor domain being present. The coding sequence is preferably in sense-orientation and encodes a desired, biologically active protein or peptide, or an active peptide fragment. In gene silencing approaches, the DNA sequence is preferably present in the form of an antisense DNA or an inverted repeat DNA, comprising a short sequence of the target gene in antisense, or in sense and antisense orientation. “Ectopic expression” refers to expression in a tissue in which the gene is normally not expressed.

A “transcription regulatory sequence” is herein defined as a nucleic acid sequence that is capable of regulating the rate of transcription of a (coding) sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is essentially only active in specific types of tissues or cells.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which a joined to form a functional protein, which displays the functionality of the joined domains (for example DNA binding or repression leading to a dominant negative function). A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain. Specific domains can also be used to identify protein members belonging to the AtCHR12 genes, such as AtCHR12 orthologs from other plant species. Examples of domains found in ATCHR12 proteins are the SNF2 family N-terminal domain (Pfam PF00176) and the Helicase conserved C-terminal domain (Pfam PF00271).

The terms “target peptide” refers to amino acid sequences which target a protein to intracellular organelles such as plastids, preferably chloroplasts, mitochondria, or to the extracellular space (secretion signal peptide). A nucleic acid sequence encoding a target peptide may be fused (in frame) to the nucleic acid sequence encoding the amino terminal end (N-terminal end) of the protein.

A “nucleic acid construct” or “vector” is herein understood to mean a man-made nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US2002138879 and WO9506722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors usually comprise further genetic elements to facilitate their use in molecular cloning, such as e.g. selectable markers, multiple cloning sites and the like (see below).

A “host cell” or a “recombinant host cell” or “transformed cell” are terms referring to a new individual cell (or organism) arising as a result of at least one nucleic acid molecule, especially comprising a chimeric gene encoding a desired protein or a nucleic acid sequence which upon transcription yields an antisense RNA or an inverted repeat RNA (dsRNA or hairpin RNA) for silencing of a target gene/gene family, having been introduced into said cell. The host cell is preferably a plant cell or a bacterial cell. The host cell may contain the nucleic acid construct as an extra-chromosomally (episomal) replicating molecule, or more preferably, comprises the chimeric gene integrated in the nuclear or plastid genome of the host cell.

The term “selectable marker” is a term familiar to one of ordinary skill in the art and is used herein to describe any genetic entity which, when expressed, can be used to select for a cell or cells containing the selectable marker. Selectable marker gene products confer for example antibiotic resistance, or more preferably, herbicide resistance or another selectable trait such as a phenotypic trait (e.g. a change in pigmentation) or a nutritional requirements. The term “reporter” is mainly used to refer to visible markers, such as green fluorescent protein (GFP), eGFP, luciferase, GUS and the like.

The term “ortholog” of a gene or protein refers herein to the homologous gene or protein found in another species, which has the same function as the gene or protein, but (usually) diverged in sequence from the time point on when the species harbouring the genes diverged (i.e. the genes evolved from a common ancestor by speciation). Orthologs of the AtCHR12 genes (from Arabidopsis) may thus be identified in other plant species based on both sequence comparisons (e.g. based on percentages sequence identity over the entire sequence or over specific domains) and functional analysis.

The terms “homologous” and “heterologous” refer to the relationship between a nucleic acid or amino acid sequence and its host cell or organism, especially in the context of transgenic organisms. A homologous sequence is thus naturally found in the host species (e.g. a tomato plant transformed with a tomato gene), while a heterologous sequence is not naturally found in the host cell (e.g. a tomato plant transformed with a sequence from potato plants). Depending on the context, the term “homolog” or “homologous” may alternatively refer to sequences which are descendent from a common ancestral sequence (e.g. they may be orthologs).

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“Sequence identity” and “sequence similarity” can be determined by alignment of two peptide or two nucleotide sequences using global or local alignment algorithms. Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimises the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. Alternatively percent similarity or identity may be determined by searching against databases such as FASTA, BLAST, etc.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

Whenever reference to a “plant” or “plants” (or a plurality of plants) according to the invention is made, it is understood that also plant parts (cells, tissues or organs, seeds, embryos, severed or harvested parts, leaves, seedlings, flowers, pollen, fruit, tubers, stems, roots, callus, protoplasts, etc), progeny or clonal propagations of the plants which retain the distinguishing characteristics of the parents (e.g. presence of a transgene), such as seed obtained by selfing or crossing, e.g. hybrid seed (obtained by crossing two inbred parental lines), hybrid plants and plant parts derived therefrom are encompassed herein, unless otherwise indicated.

### DETAILED DESCRIPTION

The present inventors found that plant growth can be modified using chromatin remodeling genes, such as the Arabidopsis AtCHR12 gene, or homologs or orthologs thereof. Especially, transgenic plants comprising these genes have temporarily modified growth, especially under one or more stress conditions. In addition, non-transgenic crop plants comprising one or more AtCHR12 genes introduced from other plants (i.e. either wild type alleles or natural or induced mutant alleles) are encompassed herein, as are methods for generating these. Such plants have the benefit of not falling under the GMO regulations, while having the novel phenotypes conferred by the chromatin remodeling alleles introduced by breeding (e.g. marker assisted selection, MAS).

Under non-stress growing conditions only few plants over-expressing the AtCHR12 gene (11-fold), encoding the protein of SEQ ID NO: 1, showed temporal growth arrest of the primary inflorescence. This phenotype was rare, unstable and unpredictable, and likely associated with some unintended residual stress exposure (which is unavoidable in experimental settings). Thus, the majority of plants showed completely normal growth under non-stress conditions. However, when investigating growth of these AtCHR12 overexpressing plants under various stress conditions (heat, drought) it was surprisingly found that stress significantly enhanced inflorescence growth arrest of over-expressing plants compared to wild type plants exposed to the same stress conditions. It was further found that knock-down mutants were less inhibited in growth than wild type or over-expressing plants under stress conditions (salinity), i.e. their growth was comparable to growth of wild type plants under non-stress conditions.

Based on the above findings the involvement of chromatin remodeling genes in growth modulation can be exploited in various ways as described herein below.

### Nucleic Acid and Amino Acid Sequences According to the Invention

In one embodiment of the invention any nucleic acid sequence encoding a ATCHR12 protein, or protein variant, or protein fragment, may be used for making a chimeric gene, vector and transformed plant or plant cell, either using an expression vector or a gene silencing vector, as described further below.

Any AtCHR12 nucleic acid sequence (cDNA, genomic DNA, RNA) encoding a ATCHR12 protein or protein fragment may be used (referred herein to as “AtCHR12 nucleic acid sequence”). A “ATCHR12 protein” or a “CHR12 protein” refers to a protein which is essentially similar to SEQ ID NO: 1, i.e. it comprises at least 40, 50, 60, 70, 80, 90, 95, 97, 98, 99% or more amino acid sequence identity to SEQ ID NO: 1 (depicting the Arabidopsis ATCHR12 protein) when aligned over the entire length, or fragments of any of these. Due to the degeneracy of the genetic code, various nucleic acid sequences encode the same protein, and are thus encompassed herein. For example, the nucleic acid sequences of SEQ ID NO: 2-4 encode the ATCHR12 protein of SEQ ID NO: 1. Nucleic acid sequences encoding a ATCHR12 protein may be isolated from various sources or made synthetically, as described below.

Also included are variants and fragments of AtCHR12 nucleic acid sequences, such as nucleic acid sequences hybridizing to AtCHR12 nucleic acid sequences (e.g. to SEQ ID NO: 2-4) under stringent hybridization conditions as defined. Variants of AtCHR12 nucleic acid sequences include nucleic acid sequences which have a nucleic acid sequence identity to any one of SEQ ID NO: 2 to 4 (AtCHR12) of at least 50% or more, preferably at least 55%, 60%, 70%, 80%, 90%, 95%, 99%, 99.5%, 99.8% or more, as determined using pairwise alignment using the GAP program using full lengths sequences. Such variants may also be referred to as being “essentially similar” to any one of SEQ ID NO: 2-4. Fragments include parts of any of the above ATCHR12 nucleic acid sequences (or variants), which may for example be used as primers or probes or in gene silencing constructs. Parts may be contiguous stretches of at least 10, 15, 19, 20, 21, 22, 23, 25, 50, 100, 200, 450, 500, 1000 or more nucleotides in length. Preferably the AtCHR12 nucleic acid sequences are of plant origin (i.e. they naturally occur in plant species) or are modified plant sequences.

It is clear that many methods can be used to identify, synthesise or isolate variants or fragments of AtCHR12 nucleic acid sequences, such as nucleic acid hybridization, PCR technology, in silico analysis and nucleic acid synthesis, and the like. Thus, an ATCHR12-protein encoding nucleic acid sequence may be a sequence which is chemically synthesized or which is cloned from any organism (e.g. plant, animal, fungi, yeast), but preferably plant sequences are used, more preferably a sequence originating from a particular plant species is reintroduced into said species (optionally with prior sequence modification, such as codon usage optimization). Thus, in a preferred embodiment, the CHR12 DNA corresponds to, or is a modification/variant of, the endogenous CHR12 DNA of the species which is used as host species in transformation. Thus, a potato CHR12 cDNA or genomic DNA (or a variant or fragment thereof) is preferably used to transform potato plants. Most preferably (for regulatory approval and public acceptance reasons) the nucleic acid sequence is operably linked to a transcription regulatory sequence, especially a promoter, which also originates from a plant or even from the same plant which is to be transformed.

An active or functional ATCHR12 protein or variant or fragment is a protein or peptide which shows activity in the cell in vivo, i.e. it has biological activity and is therefore able to modify growth of one or more tissues or organs of a transformed plant.

Biological activity (or biological function) can be tested using a variety of known methods, for example by generating a transformed plant over-expressing the gene as described in the Examples and analyzing whether a change growth rate and/or growth period of one or more tissues or organs is measurable, for example when the plant or plant part is exposed to stress. The effect on growth arrest can suitably be compared to either non-transformed or empty vector transformed controls, or to transformants expressing a nucleic acid sequence encoding the protein of SEQ ID NO: 1.

Biological activity may also be determined by assaying other functionalities of the protein (or variant or fragment), such as its capability to hydrolyse ATP, to disrupt histone-DNA interaction or to induce expression of one or more dormancy-associated genes, as described in the Examples.

It is understood that in any transformation experiments a certain degree of variation in the phenotype of transformants is seen, normally due to position effects in the genome and/or due to copy number. A skilled person will know how to compare transformants to one another, e.g. by selecting single copy number events and analysing these. Other methods of determining or confirming in vivo gene/protein function include the generation of knock-out mutants or transient expression studies. Promoter-reporter gene expression studies may also provide information as to the spatio-temporal expression pattern and the role of the protein, as described in the Examples.

The ATCHR12 protein from Arabidopsis is provided herein, but other plant homologs or orthologs can be isolated using routine methods and their functionality tested. Due to the degeneracy of the genetic code, additional nucleic acid sequences encoding the protein of SEQ ID NO: 1 are also provided. These sequences, as well as variants and fragments encoding functional CHR12 proteins, are used in a preferred embodiment, especially for making expression constructs and for the transformation of crop plants, such as root or tuber crops or bulbs.

Other putative CHR12 encoding nucleic acid sequences can be identified in silico, e.g. by identifying nucleic acid or protein sequences in existing nucleic acid or protein database (e.g. GENBANK, SWISSPROT, TrEMBL) and using standard sequence analysis software, such as sequence similarity search tools (BLASTN, BLASTP, BLASTX, TBLAST, FASTA, etc.). Especially the screening of plant sequence databases, such as the wheat genome database, etc. for the presence of amino acid sequences or nucleic acid sequences encoding CHR12 proteins is desired. Putative amino acid sequences or nucleic acid sequences can then be selected, cloned or synthesized de novo and tested for in vivo functionality by e.g. over-expression in a host or host cell. Further sequences may be identified using known AtCHR12 sequences to design (degenerate) primers or probes as described below.

For optimized in-planta expression the codon usage of an AtCHR12-encoding nucleic acid sequence is, in one embodiment, adapted to the preferred codon usage of the host species which is to be transformed. In a preferred embodiment any of the above AtCHR12 DNA sequences (or variants) are codon-optimized by adapting the codon usage to that most preferred in the host genus or preferably the host species (Bennetzen & Hall, 1982, J. Biol. Chem. 257, 3026-3031; Itakura et al., 1977 Science 198, 1056-1063.) using available codon usage tables (e.g. more adapted towards expression in potato, cotton, soybean corn or rice). Codon usage tables for various plant species are published for example by Ikemura (1993, In “Plant Molecular Biology Labfax”, Croy, ed., Bios Scientific Publishers Ltd.) and Nakamura et al. (2000, Nucl. Acids Res. 28, 292.) and in the major DNA sequence databases (e.g. EMBL at Heidelberg, Germany). Accordingly, synthetic DNA sequences can be constructed so that the same or substantially the same proteins are produced. Several techniques for modifying the codon usage to that preferred by the host cells can be found in patent and scientific literature. The exact method of codon usage modification is not critical for this invention. Other modification, which may optimize expression in plants and/or which make cloning procedures easier may be carried out, such as removal of cryptic splice sites, avoiding long AT or GC rich stretches, etc. (see Examples). Such methods are known in the art and standard molecular biology techniques can be used. A “codon-optimized” sequence preferably has at least about the same GC content or a higher GC content than the genes of the host species into which it is to be introduced. For example, in L. esculentum the GC content of endogenous genes is about 30-40%. The preferred GC contents of CHR12-encoding nucleic acid sequences for transformation of L. esculentum is therefore a GC content of at least 30-40%, preferably above 40%, such as at least 45%, 50%, 55%, 60%, 70% or more. Preferably regions of very high (>80%) or very low (<30%) GC content should be avoided.

Small modifications to a DNA sequence can be routinely made, i.e., by PCR-mediated mutagenesis (Ho et al., 1989, Gene 77, 51-59., White et al., 1989, Trends in Genet. 5, 185-189). More profound modifications to a DNA sequence can be routinely done by de novo DNA synthesis of a desired coding region using available techniques.

Also, the CHR12 nucleic acid sequences can be modified so that the N-terminus of the CHR12 protein has an optimum translation initiation context, by adding or deleting one or more amino acids at the N-terminal end of the protein. Often it is preferred that the proteins of the invention to be expressed in plants cells start with a Met-Asp or Met-Ala dipeptide for optimal translation initiation. An Asp or Ala codon may thus be inserted following the existing Met, or the second codon, Val, can be replaced by a codon for Asp (GAT or GAC) or Ala (GCT, GCC, GCA or GCG). The DNA sequences may also be modified to remove illegitimate splice sites.

As mentioned above CHR12 proteins can be (in addition to their function) defined structurally by the percentage sequence identity over their entire length. CHR12 proteins have a sequence identity of at least 40% or more over their entire length to SEQ ID NO: 1 (ATCHR12), such as but not limited to at least 43%, 45%, 50%, 55%, 56%, 58%, 60%, 70%, 75%, 80%, 85%, 90%, 95%, 98%, 99%, 99.5,%, 99.8% or more at the amino acid sequence level, as determined using pairwise alignment using the GAP program (with a gap creation penalty of 8 and an extension penalty of 2). Such variants may also be referred to as being “essentially similar” to SEQ ID NO: 1. Preferably proteins having some, preferably 5-10, 20, 30, 50, 100, 200, 300, or more amino acids added, replaced or deleted without significantly changing the protein activity are included in this definition. For example conservative amino acid substitutions within the categories basic (e.g. Arg, His, Lys), acidic (e.g. Asp, Glu), nonpolar (e.g. Ala, Val, Trp, Leu, Ile, Pro, Met, Phe, Trp) or polar (e.g. Gly, Ser, Thr, Tyr, Cys, Asn, Gln) fall within the scope of the invention as long as the activity of the CHR12 protein is not significantly, preferably not, changed or at least not reduced, e.g. when compared with the activity of SEQ ID NO: 1. In addition non-conservative amino acid substitutions fall within the scope of the invention as long as the activity of the CHR12 protein is not changed significantly, preferably not changed or at least not reduced, e.g. when compared with the activity of SEQ ID NO: 1. Also CHR12 protein fragments and active chimeric CHR12 proteins are encompassed herein. Protein fragments may for example be used to generate antibodies against CHR12 (anti-CHR12 antibodies), as described elsewhere herein. Protein fragments may be fragments of at least about 5, 10, 20, 40, 50, 60, 70, 90, 100, 150, 152, 160, 200, 220, 230, 250, 300, 400, 500, 600, 700 or more contiguous amino acids. Nucleic acid sequences encoding such fragments are also provided, which may for example be used in the construction of gene silencing vectors as described below or for the expression of peptides which can be used to raise antibodies against. Also, the smallest protein fragment which retains activity in vivo in plants is also provided. A nucleic acid sequence encoding such a fragment may be use to generate a transgenic plant as described.

### Chimeric Genes and Vectors According to the Invention

### Expression Vectors

In one embodiment of the invention nucleic acid sequences encoding CHR12 proteins (or variants or fragments) as described above, are used to make chimeric genes, and vectors comprising these for transfer of the chimeric gene into a host cell and production of the CHR12 protein in host cells, such as cells, tissues, organs or whole organisms derived from transformed cell(s).

Host cells are preferably plant cells. Any plant may be a suitable host, such as monocotyledonous plants or dicotyledonous plants, for example maize/corn (Zea species, e.g. Z. mays, Z. diploperennis (chapule), Zea luxurians (Guatemalan teosinte), Zea mays subsp. huehuetenangensis (San Antonio Huista teosinte), Z. mays subsp. mexicana (Mexican teosinte), Z. mays subsp. parviglumis (Balsas teosinte), Z. perennis (perennial teosinte) and Z. ramosa), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), tobacco (Nicotiana species), alfalfa (Medicago sativa), rice (Oryza species, e.g. O. sativa indica cultivar-group or japonica cultivar-group), forage grasses, pearl millet (Pennisetum spp. e.g. P. glaucum), tree species, vegetable species, such as Lycopersicon ssp (recently reclassified as belonging to the genus Solanum), e.g. tomato (L. esculentum, syn. Solanum lycopersicum), potato (Solanum tuberosum) and other Solanum species, such as eggplant (Solanum melongena), tomato (S. lycopersicum, e.g. cherry tomato, var. cerasiforme or current tomato, var. pimpinellifolium), tree tomato (S. betaceum, syn. Cyphomandra betaceae), pepino (S. muricatum), cocona (S. sessiliflorum) and naranjilla (S. quitoense); peppers (Capsicum annuum, Capsicum frutescens), pea (e.g. Pisum sativum), bean (e.g. Phaseolus species), fleshy fruit (grapes, peaches, plums, strawberry, mango) ornamental species (e.g. Rose, Petunia, Chrysanthemum, Lily, Gerbera species), woody trees (e.g. species of Populus, Salix, Quercus, Eucalyptus), fibre species e.g. flax (Linum usitatissimum) and hemp (Cannabis sativa). In one embodiment vegetable species, especially Solanum species (including Lycopersicon species) are preferred.

In one embodiment particularly root vegetable and tuber species and bulb producing species are preferred. Most preferred root vegetable species are sugar beet (Beta vulgaris), Brassica species (e.g. rutabaga and turnip), carrot (Daucus carota), celeriac (Apium graveolens), potato (Solarium tuberosum), sweet potato (Ipomoea batatas), cassaya (Manihot esculenta), taro (Colocasia esculenta), radish (Raphanus sativus), yam (Dioscorea spp), artichoke (Helianthus tuberosus and Stachys affinis).

In another embodiment early bolting/early flowering species or “bolting susceptible” species are preferred, such as lettuce, Brassica (e.g. Brassica oleracea, B. napus), sugar beet, onion, carrot, celery, potato, etc. (see also further below). Thus, for example species of the following genera may be transformed: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Cucumis, Hyoscyamus, Lycopersicon, Nicotiana, Solanum, Malus, Petunia, Digitalis, Majorana, Ciahorium, Helianthus, Lactuca, Bromus, Citrullus, Asparagus, Antirrhinum, Heterocallis, Nemesis, Pelargonium, Panieum, Pennisetum, Ranunculus, Senecio, Salpiglossis, Browaalia, Glycine, Pisum, Phaseolus, Gossypium, Glycine and Lolium.

The construction of chimeric genes and vectors for introduction of CHR12-protein encoding nucleic acid sequences into the genome of host cells is generally known in the art. To generate a chimeric gene the nucleic acid sequence encoding a CHR12 protein (or variant or functional fragment) is operably linked to a promoter sequence, suitable for expression in the host cells, using standard molecular biology techniques. The promoter sequence may already be present in a vector so that the CHR12 nucleic sequence is simply inserted into the vector downstream of the promoter sequence. The vector is then used to transform the host cells and the chimeric gene is inserted in the nuclear genome and expressed there using a suitable promoter (e.g., Mc Bride et al., 1995 Bio/Technology 13, 362; U.S. Pat. No. 5,693,507). In one embodiment a chimeric gene comprises a suitable promoter for expression in plant cells, operably linked thereto a nucleic acid sequence encoding a CHR12 protein, protein variant or protein fragment (or fusion protein or chimeric protein) according to the invention, optionally followed by a 3′nontranslated nucleic acid sequence. The promoter specificity is thought not to be crucial to the invention, as the CHR12 chromatin remodelling gene will modify growth upon stress exposure, as long as the promoter is (sufficiently) active in the host cells and sufficient recombinant CHR12 protein is produced. For example, when the constitutive CaMV 35S promoter was used, the growth arrest phenotype was observed after stress exposure. Therefore, a wide range of promoters which are active in plant cells can suitably be used.

The CHR12 nucleic acid sequence, preferably the CHR12 chimeric gene, encoding an functional CHR12 protein (or fragment or variant), can be stably inserted in a conventional manner into the nuclear genome of a single plant cell, and the so-transformed plant cell can be used in a conventional manner to produce a transformed plant that has an altered phenotype due to the presence of the CHR12 protein in certain cells at a certain time. In this regard, a T-DNA vector, comprising a nucleic acid sequence encoding an CHR12 protein, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95, 426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718.

Preferred T-DNA vectors each contain a promoter operably linked to CHR12 encoding nucleic acid sequence between T-DNA border sequences, or at least located to the left of the right border sequence. Border sequences are described in Gielen et al. (1984, EMBO J 3, 835-845). Of course, other types of vectors can be used to transform the plant cell, using procedures such as direct gene transfer (as described, for example in EP 0 223 247, or particle or micro-projectile bombardment as described in US2005/055740 and WO2004/092345), pollen mediated transformation (as described, for example in EP 0 270 356 and WO85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as those described methods for transforming certain lines of corn (e.g., U.S. Pat. No. 6,140,553; Fromm et al., 1990, Bio/Technology 8, 833-839; Gordon-Kamm et al., 1990, The Plant Cell 2, 603-618) and rice (Shimamoto et al., 1989, Nature 338, 274-276; Datta et al. 1990, Bio/Technology 8, 736-740) and the method for transforming monocots generally (PCT publication WO92/09696). For cotton transformation see also WO 00/71733, and for rice transformation see also the methods described in WO92/09696, WO94/00977 and WO95/06722. For sorghum transformation see e.g. Jeoung J M et al. 2002, Hereditas 137: 20-8 or Zhao Z Y et al. 2000, Plant Mol Biol. 44:789-98). Likewise, selection and regeneration of transformed plants from transformed cells is well known in the art. Obviously, for different species and even for different varieties or cultivars of a single species, protocols are specifically adapted for regenerating transformants at high frequency.

The resulting transformed plant can be used in a conventional plant breeding scheme to produce either more transformed plants containing the transgene or to produce recombinant plants/plant populations, preferably lacking the chimeric gene.

The CHR12 nucleic acid sequence is inserted in a plant cell genome so that the inserted coding sequence is downstream (i.e. 3′) of, and under the control of, a promoter which can direct the expression in the plant cell. This is preferably accomplished by inserting the chimeric gene in the plant cell genome, particularly in the nuclear genome.

Preferred promoters include promoters which are active at least during one or more stages of plant growth and/or development.

Suitable promoters are the promoters of CHR12 genes themselves or from CHR12 homologous or orthologous genes. For example, the AtCHR12 promoter may be used (see SEQ ID NO: 5), or functional fragments thereof. Equally, the promoters of any other CHR12 gene, especially of plant origin, may be used. Functional fragments of promoters, such as SEQ ID NO: 5, can be obtained by deletion-analysis combined with (transient) expression analysis, as known in the art. The promoter of SEQ ID NO: 5, and variants thereof (especially nucleic acid sequences comprising at least 70, 80, 90, 95, 98, 99% or more sequence identity to SEQ ID NO: 5), as well as functional fragments of any of these, are also embodiments of the invention. Also chimeric genes and vectors comprising SEQ ID NO: 5 or variants or fragments thereof, as well as plant cells and plant parts comprising these, are encompassed herein.

Alternatively, the CHR12-encoding nucleic acid sequence may be placed under the control of an inducible promoter. Examples of inducible promoters are the Adhl promoter which is inducible by hypoxia or cold stress, the Hsp70 promoter which is inducible by heat stress, and the PPDK promoter which is inducible by light. Other examples of inducible promoters are wound-inducible promoters, such as the MPI promoter described by Cordera et al. (1994, The Plant Journal 6, 141), which is induced by wounding (such as caused by insect or physical wounding), or the COMPTII promoter (WO0056897) or the promoter described in U.S. Pat. No. 6,031,151. Alternatively the promoter may be inducible by a chemical, such as dexamethasone as described by Aoyama and Chua (1997, Plant Journal 11: 605-612) and in U.S. Pat. No. 6,063,985 or by tetracycline (TOPFREE or TOP 10 promoter, see Gatz, 1997, Annu Rev Plant Physiol Plant Mol Biol. 48: 89-108 and Love et al. 2000, Plant J. 21: 579-88). Other inducible promoters are for example inducible by a change in temperature, such as the heat shock promoter described in U.S. Pat. No. 5,447,858, by anaerobic conditions (e.g. the maize ADHIS promoter), by light (U.S. Pat. No. 6,455,760) and e.g. the potato Lhca3.St.1 promoter (Nap, J. P. et al., 1993, Plant Mol Biol 23, 605-612), etc. One preferred promoter is the ethanol-inducible promoter system, as described in Ait-ali et al. (2001, Plant Biotechnology Journal 1, 337-343), wherein ethanol treatment activates alcR, which in turn induces expression of the alc:35S promoter.

Obviously, there are a range of other promoters available. Examples of promoters under developmental control include the anther specific promoter 5126 (U.S. Pat. Nos. 5,689,049 and 5,689,051), glob-1 promoter, and gamma-zein promoter. Similarly tissue specific or tissue preferred promoters may be used, such as promoters mainly active in leaves, tubers, roots, stems, bulbs, green tissue, fruit, seed, anthers, inflorescences, etc.

Constitutive promoters may also be used in certain embodiments. Suitable constitutive promoters include: the strong constitutive 35S promoters or enhanced 35S promoters (the “35S promoters”) of the cauliflower mosaic virus (CaMV) of isolates CM 1841 (Gardner et al., 1981, Nucleic Acids Research 9, 2871-2887), CabbB-S (Franck et al., 1980, Cell 21, 285-294) and CabbB-JI (Hull and Howell, 1987, Virology 86, 482-493); the 35S promoter described by Odell et al. (1985, Nature 313, 810-812) or in U.S. Pat. No. 5,164,316, promoters from the ubiquitin family (e.g. the maize ubiquitin promoter of Christensen et al., 1992, Plant Mol. Biol. 18, 675-689, EP 0 342 926, see also Cornejo et al. 1993, Plant Mol. Biol. 23, 567-581), the gos2 promoter (de Pater et al., 1992 Plant J. 2, 834-844), the emu promoter (Last et al., 1990, Theor. Appl. Genet. 81, 581-588), Arabidopsis actin promoters such as the promoter described by An et al. (1996, Plant J. 10, 107.), rice actin promoters such as the promoter described by Zhang et al. (1991, The Plant Cell 3, 1155-1165) and the promoter described in U.S. Pat. No. 5,641,876 or the rice actin 2 promoter as described in WO070067; promoters of the Cassaya vein mosaic virus (WO 97/48819, Verdaguer et al. 1998, Plant Mol. Biol. 37, 1055-1067), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′promoter” and “TR2′promoter”, respectively) which drive the expression of the 1′ and 2′ genes, respectively, of the T-DNA (Velten et al., 1984, EMBO J 3, 2723-2730), the Figwort Mosaic Virus promoter described in U.S. Pat. No. 6,051,753 and in EP426641, histone gene promoters, such as the Ph4a748 promoter from Arabidopsis (PMB 8: 179-191), Smas promoter, the cinnamyl alcohol dehydrogenase promoter (U.S. Pat. No. 5,683,439), the Nos promoter, the pEmu promoter, the rubisco promoter, the GRP1-8 promoter or others.

The CHR12 coding sequence is inserted into the plant genome so that the coding sequence is upstream (i.e. 5′) of suitable 3′end transcription regulation signals (“3′end”) (i.e. transcript formation and polyadenylation signals). Polyadenylation and transcript formation signals include those of, the nopaline synthase gene (“3′ nos”) (Depicker et al., 1982 J. Molec. Appl. Genetics 1, 561-573.), the octopine synthase gene (“3′ocs”) (Gielen et al., 1984, EMBO J 3, 835-845) and the T-DNA gene 7 (“3′ gene 7”) (Velten and Schell, 1985, Nucleic Acids Research 13, 6981-6998), which act as 3′-untranslated DNA sequences in transformed plant cells, and others.

A CHR12-encoding nucleic acid sequence can optionally be inserted in the plant genome as a hybrid gene sequence whereby the CHR12 sequence is linked in-frame to a (U.S. Pat. No. 5,254,799; Vaeck et al., 1987, Nature 328, 33-37) gene encoding a selectable or scorable marker, such as for example the neo (or nptII) gene (EP 0 242 236) encoding kanamycin resistance, so that the plant expresses a fusion protein which is easily detectable.

For obtaining enhanced expression in monocot plants such as grass species, e.g. corn or rice, an intron, preferably a monocot intron, can be added to the chimeric gene. For example the insertion of the intron of the maize Adhl gene into the 5′ regulatory region has been shown to enhance expression in maize (Callis et. al., 1987, Genes Develop. 1: 1183-1200). Likewise, the HSP70 intron, as described in U.S. Pat. No. 5,859,347, may be used to enhance expression. The DNA sequence of the CHR12 nucleic acid sequence can be further changed in a translationally neutral manner, to modify possibly inhibiting DNA sequences present in the gene part by means of site-directed intron insertion and/or by introducing changes to the codon usage, e.g., adapting the codon usage to that most preferred by plants, preferably the specific relevant plant genus or species, as described above.

### Gene Silencing Vectors

For certain applications, such as reducing stress-induced growth arrest (or retardation), it is desired to generate transgenic plants in which the endogenous CHR12 gene or the CHR12 gene family is non-functional (T-DNA insertion, mutation), silenced or is silenced in specific cells or tissues of the plant.

The embodiments described above, for methods of making transgenic plants which over-express an CHR12 protein, also apply to methods for making transgenic plants wherein endogenous CHR12 gene(s) is/are silenced, with the difference that gene silencing vectors are used. “Gene silencing” refers to the down-regulation or complete inhibition of gene expression of one or more target genes. The use of inhibitory RNA to reduce or abolish gene expression is well established in the art and is the subject of several reviews (e.g Baulcombe 1996, Plant Cell 8: 1833-1844; Stam et al. 1997, Ann. Botan. 79: 3-12; Depicker and Van Montagu, 1997, Curr. Opin. Cell. Biol. 9: 373-382). There are a number of technologies available to achieve gene silencing in plants, such as chimeric genes which produce antisense RNA of all or part of the target gene (see e.g. EP 0140308 B1, EP 0240208 B1 and EP 0223399 B1), or which produce sense RNA (also referred to as co-suppression), see EP 0465572 B1.

The most successful approach so far has however been the production of both sense and antisense RNA of the target gene (“inverted repeats”), which forms double stranded RNA (dsRNA) in the cell and silences the target gene. Methods and vectors for dsRNA production and gene silencing have been described in EP 1068311, EP 983370 A1, EP 1042462 A1, EP 1071762 A1 and EP 1080208 A1.

A vector according to the invention may therefore comprise a transcription regulatory region which is active in plant cells operably linked to a sense and/or antisense DNA fragment of a CHR12 gene according to the invention. Generally short (sense and antisense) stretches of the target gene sequence, such as 17, 18, 19, 20, 21, 22, 23, 24 or 25 nucleotides of coding or non-coding sequence are sufficient. Longer sequences can also be used, such as 50, 100, 200 or 250 nucleotides, or more. Preferably, the short sense and antisense fragments are separated by a spacer sequence, such as an intron, which forms a loop (or hairpin) upon dsRNA formation. Any short stretch of contiguous nucleotides of any one of SEQ ID NO: 2-4, or of a variant thereof, may be used to make a CHR12 gene silencing vector and a transgenic plant in which one or more CHR12 genes are silenced in all or some tissues or organs or at a certain developmental stage. A convenient way of generating hairpin constructs is to use generic vectors such as pHANNIBAL and pHELLSGATE, vectors based on the Gateway® technology (see Wesley et al. 2004, Methods Mol Biol. 265:117-30; Wesley et al. 2003, Methods Mol Biol. 236:273-86 and Helliwell & Waterhouse 2003, Methods 30(4):289-95.), all incorporated herein by reference.

By choosing conserved nucleic acid sequences all CHR12 gene family members in a host plant can be silenced. Encompassed herein are also transgenic plants comprising a promoter active in plants, operably linked to a sense and/or antisense DNA fragment of a CHR12 nucleic acid sequence and exhibiting a CHR12 gene silencing phenotype (a significant alteration in growth, as described further below).

In both application, the chimeric gene may be introduced stably into the host genome or may be present as an episomal unit.

### Transgenic Plants and Plant Parts According to the Invention

Transgenic plants, plant cells, tissues or organs are provided, obtainable by the above methods. These plants are characterized by the presence of a chimeric gene in their cells or genome and/or by having modified growth during one or more developmental stages and/or environmental conditions, compared to the non-transgenic (wild type) controls or compared to empty vector controls.

Growth of one or more tissues (or whole plants) can most easily be measured by visual assessment of a plurality of plants or tissues at regular time intervals, e.g. by measuring plant height, internode length, leaf dimensions, etc. Obviously, also other methods for measuring and optionally quantifying growth and growth rate (growth/time) can also be used.

Transformants expressing high, moderate or low levels of the CHR12 protein (or of the sense and/or antisense transcript in silenced plants) can be selected by e.g. analysing copy number (Southern blot analysis), mRNA transcript levels (e.g. Northern blot analysis or RT-PCR using AtCHR12 primer pairs or flanking primers) or by analysing the presence and level of CHR12 protein (e.g. SDS-PAGE followed by Western blot analysis; ELISA assays, immunocytological assays, etc). The expression level of the CHR12 chimeric gene will depend not only on the strength and specificity of the promoter, but also on the position of the chimeric gene in the genome. The strength of the growth modification will, therefore also vary in a dosage dependent manner. The skilled person can select those transformants which show the most useful modification in growth. For example, by testing various promoters and analyzing a variety of recombinant plants transformed with the same construct (i.e. “transformation events”), the desired transformant, having the desired level of growth modification or growth rate, can be identified and selected for further use. The same applies for plants transformed with a gene silencing construct, where a suitable construct and transformation event can easily be selected using routine methods.

Thus, in one embodiment of the invention a transgenic plant or plant part comprising a chimeric gene integrated in its genome is provided, characterized in that said chimeric gene comprises a transcription regulatory sequence active in plant cells operably linked to a nucleic acid sequence selected from the group consisting of:

(a) a nucleic acid sequence encoding a protein of SEQ ID NO: 1;
(b) a nucleic acid sequence encoding a protein having at least 40, 50, 60, 70% or more amino acid identity to SEQ ID NO: 1 over the entire length;
(c) a sense and/or antisense fragment of the sequence of (a) or (b).

Preferably, the transgenic plant, or plant part, is modified in at least one growth characteristic compared to a suitable control.

The plants and plant parts expressing a functional CHR12 protein or protein fragment comprise significant (temporal and stress-dependent) growth arrest or growth retardation of one or more tissues or organs, depending on the gene dosage, promoter and position in the genome. Transformation events having the optimal modified growth can be made and identified (e.g. by testing various gene dosages or copy number effects, promoters and selecting events having the desired phenotype under stress and/or non-stress conditions).

In one embodiment growth of one or more of the following tissues is arrested during one or more growing conditions (especially temporal arrest when exposed to one or more stress conditions, while growth continues once the stress is removed or lowered):

(a) the main stern, leading to plants of reduced height, such as plants which are at least 10%, preferably at least 15, 20, 30, 40 50, 60, 70, 80% (or more) reduced in height relative to a suitable control plant (e.g. a wild type plant) when grown under the same conditions (e.g. under one or more stress conditions); transgenic plants which temporarily are arrested or slowed down in growth during stress are useful for predetermining and delaying harvest time, a prolonging the harvest period and/or increasing yield and/or survival in stress exposed locations (such as wind exposed or water deficient locations).
(b) the primary inflorescence, leading to plants having a reduced height of the primary inflorescence, such as plants which are at least 10%, preferably at least 15, 20, 30, 40 50, 60, 70, 80% (or more) reduced in inflorescence height relative to a suitable control plant (e.g. a wild type plant) when grown under the same conditions; in addition the axillary shoots may overgrow the primary shoot and/or increase in number; similarly, the number of inflorescences may increase on axillary shoots; advantages of such plants are as under (a). This phenotype is of interest for producing dwarf ornamental plants (e.g. cut flowers) or altering the harvest time point of (normally grown, but developmentally delayed) ornamentals, such as flowers.
(c) the buds, especially the buds of (harvested) underground storage organs, such as potato tubers, leading to a prolonged and/or more uniform dormancy period; especially post-harvest sprouting can be delayed by one or more weeks or months. For more general terms, see (d).
(d) the sprouting of any geophyte (underground storage organs, including modified rhizomes or stems, such as tubers, underground stems such as corms and underground shoots such as bulbs, e.g. onions); harvested transgenic storage organs can thereby be stored for longer periods without sprouting and/or uniformity of sprouting can be increased. Depending on the plant species and variety or line, the harvested storage organs (e.g. potato tuber batches) can be exposed to one or more stresses, such as cold temperature, to uniformly inhibit sprouting for longer periods.
(e) the bolting (or premature flowering) of plants can be delayed or prevented entirely. Thus by expressing a CHR12 protein according to the invention, so-called bolting-resistant or bolting delayed plants can be made. For example, a plant which is normally bolting sensitive type (i.e. which responds easily to environmental cues which promote bolting) can be transformed to change the sensitivity of the plant to environmental cues which promote bolting. The transgenic plant will, therefore, not bolt at all or bolt later when exposed to these cues (such as day length and temperature). Alternatively, the plant will bolt and the seed-shoot (or inflorescence shoot) produced will die off after exposure to stress.
(f) embryo growth can be arrested and seed dormancy can be increased. By expressing CHR12 protein according to the invention a plant whose seeds would normally have a certain level of seed dormancy can be transformed to increase dormancy strength (the percentage of seeds germinating is decreased relative to the wild type by at least 10, 20, 30, 40, 50% or more) and/or decrease the temperature range under which dormancy seen in non-transgenic plants. Also, secondary dormancy can be increased and in the case of secondary dormancy of transgenic overexpressing plants the dormancy can be broken at a higher efficiency that of wild type plants. This has advantages in generating a higher, more uniform dormancy break of transgenic seeds compared to wild type seeds through for example stratification.

Preferably, the growth arrest is reversibly controlled by exposure to one or more biotic and/or abiotic stress conditions, and growth is resumed when the stress is eliminated. In this way, transgenic plants show a modified growth, especially a reduced growth as indicated above, during the exposure to stress, such as cold temperatures, hot temperatures, wind, salinity, etc. Which type of conditions are considered to be “stress” depends on the physiology of the plant. For example plants adapted to temperate climate will experience stress when the temperature conditions deviate therefrom. By switching to a dormancy-like growth arrest under stress conditions, the plants conserve energy, leading to the ability to surviving longer stress periods (increase in the percentage survival) and/or minimize yield losses compared to e.g. wild type plants.

Therefore, it is one embodiment to provide plants capable of surviving longer spells of stress (e.g. 1, 2, 3, 4 weeks or more longer than a wild type plant) and/or where a higher percentage of plants survives the stress period and/or having the same or higher yields compared to wild type plants (preferably at least 2, 5, 8, 10% more yield).

In addition the overall life span of the plants and/or the span of the vegetative and/or reproductive phase is prolonged. This is achieved due to the fact that the growth arrest is stress dependent and temporal. The plant tissue which is arrested during stress, resumes normal growth and continues growth for the same period of time as it would have done without the arrest. Thus, if there is a growth arrest for 2 or 3 weeks and the stress is removed thereafter, the development continues with a 2 or 3 week delay. The life span is therefore prolonged by 2 or 3 weeks. Overexpression of CHR12 proteins does not interfere with the plants growth and development after the stress is relieved. This has the advantages that for example the harvest time and period can be delayed by 2, 3, 4, 5, 6 or more weeks, by simply exposing the plants to one or more (mild) stress conditions (such as water deprivation) and removing the stress again when desired (e.g. by watering). One can, for example, shift harvest time to later stages of the year and thereby have a more continuous crop throughout the year.

This is, for example, particularly advantageous in the production of ornamental flowers, such as tulips, roses, etc. Therefore, in one embodiment, the transgenic plants overexpressing one or more CHR12 genes are ornamental plants.

Specific examples of transgenic plants having modified growth include for example cereals, such as rice, wheat, maize, etc., or Brassica plants having shorter main stems during exposure to stress.

Similarly, transgenic geophytes, such as potato tubers may be stored for longer periods of time, as the bud growth (sprouting) is delayed and/or made more uniform. Harvested potatoes are dormant at harvest, although the dormancy period differs significantly between cultivars and dependent on the storage conditions. For example Russet Burbanks break dormancy after 150 days at about 6° C. and at 120 days at a temperature of about 9° C. In contrast Ranger Russets break dormancy after already 75 days at about 6° C. and after 50 days at about 9° C. Storage at 4° C. or less induces cold-induced sweetening, as sugars accumulate due to starch breakdown. The present invention enables transgenic potato tubers to be stored at least 1, 2, 3, 4, 5 weeks longer, more preferably at least 2, 3, 4, 5, 6 or more months longer at a given temperature until they break dormancy, compared to non-transgenic potatoes of the same cultivar. When the potatoes are removed from storage (i.e. the cold stress is removed) they will break dormancy uniformly. Thus, premature sprouting of potato tubers can be controlled. In addition the present invention enables potato storage at higher holding temperature (1, 2, 3 degrees), more optimal for minimal respiration and yet preventing sprouting. This also lowers energy costs of potato storage.

Also transgenic plants of vegetable crops can be delayed or prevented in bolting or flowering. Premature bolting (flower initiation) in leaf vegetables such as lettuce, spinach, cabbage, rhubarb, sugarbeet, fennel, onion, carrot, etc. is a common problem for producers. Bolting is term for the beginning of flowering, when plants begin to form a seed stalk. Because vegetables are mostly grown for leaves or bulbs, rather than for seeds, their premature bolting is undesirable. Bolting is usually accompanied by toughening of the edible leaf parts as well as the redirection of nutrients away from leaves to the flowers. Bolting is mostly dependent on weather. It can be triggered by cold (vernalization; biennial crops such as onion, leek, carrot, beetroot) or changes in day length (photoperiod; annual crops such as lettuce, radish, spinach). Premature bolting is often triggered by unsettled weather conditions (cold quickly followed by warm) early in the season. Process of bolting is usually irreversible. The present invention provides transgenic plants of green vegetables with delayed or suppressed bolting, thus better for producers and consumers. See also the Examples, where bolting was arrested or the seed stalk was even destroyed by exposure to short periods of temperature stress (16 or 24 hrs, respectively). By using such transgenic plants, the need for applying chemicals which inhibit or delay bolting is reduced. Also, one can control the bolting behavior by determining the optimal time point of applying and removing stress and the optimal type and amount of stress for a given plant. Thereby, for example a bolting susceptible plant can be changed by transformation with CHR12 into a bolting resistant or bolting delayed plant. Such plants may then be grown (without bolting being initiated or with the seed-stalk being destroyed; or with bolting being delayed) under environmental conditions which would normally induce bolting (such as early spring).

In another embodiment the plants and plant parts which are silenced for CHR12 comprise a significantly less severe growth arrest of one or more tissues or organs compared to a wild type control or empty-vector control. Such plants are for example less repressed in root growth (i.e. have longer roots) or shoot growth under stress or after having experienced a period of stress. The growth phenotype of such plants under stress conditions is, thus, comparable to plants grown under non-stress conditions. Thus, especially under mild stress conditions (such as salinity, water deficiency, water logging, etc.), such plants would suffer less or no damage compared to non-transgenic plants and these plants can endure biotic and/or abiotic stress better. Such plants can therefore be advantageously grown in areas of the world which experience regular spells of stress.

Similarly, seeds which are silenced for one or more CHR12 genes may have significantly reduced (or even no) seed dormancy (embryo growth arrest). It is understood that the transgenic plants or plant parts may be homozygous or hemizygous for the transgene. In addition, other transgenes may be incorporated using known methods. Any of the transgenic plants or plant parts (fruit, tubers, leaves, flowers, etc.) described herein may be used further, e.g. in breeding schemes and the like, or for the production of food or feed products. Breeding procedures are known in the art and are described in standard text books of plant breeding, i.e., Allard, R. W., Principles of Plant Breeding (1960) New York, N.Y., Wiley, pp 485; Simmonds, N. W., Principles of Crop Improvement (1979), London, UK, Longman, pp 408; Sneep, J. et al., (1979) Tomato Breeding (p. 135-171) in: Breeding of Vegetable Crops, Mark J. Basset, (1986, editor), The Tomato crop: a scientific basis for improvement, by Atherton, J. G. & J. Rudich (editors), Plant Breeding Perspectives (1986); Fehr, Principles of Cultivar Development—Theory and Technique (1987) New York, N.Y., MacMillan.

### Nontransgenic Methods and Plants Comprising AtCHR12 Alleles

In one embodiment of the invention non-transgenic plants, especially crop plants (i.e. excluding weed species such as Arabidopsis) are provided, whereby these plants comprise one or more AtCHR12 alleles in their genome which are not naturally found in these plants and which have been introduced by either identifying wild type, functional CHR12 homologues or orthologs and/or natural or induced mutant CHR12 homologues or orthologs and by transferring these into crop species by breeding and selection (e.g. MAS), optionally using techniques such as embryo rescue, chromosome doubeling, etc.

Such functional or mutant (non-functional) alleles may originate from weed species or wild species or other plant lines of the same species, which can be crossed with the crop species. Thus, for example AtCHR12 can be transferred into other Brassicaceae crop species through interspecific hybridization or alternatively, orthologs of AtCHR12 can be identified in Brassica species such as Brassica napus (based on structure, i.e. amino acid sequence comparisons, and function compared to that of AtCHR12, using known methods such as nucleic acid hybridization; such an ortholog may then be termed BnCHR12 if it is found in Brassica napus, etc.). Functional alleles identified can be used to generate a crop species, e.g. a cultivar of Brassica napus, Brassica juncea or Brassica oleracea, which have the modified phenotypes described for transgenic AtCHR12 overexpressing plants herein. Non-functional alleles (e.g. natural mutant alleles or induced mutant alleles) can be used to generate plants having phenotypes described herein for CHR12 silencing transgenic plants.

In this way either different (functional and/or non-functional) CHR12 alleles can be introduced into a plant which naturally already comprises one or more CHR12 alleles and/or different (functional and/or non-functional) CHR12 alleles can be introduced, which are not naturally present in the plant.

It is also an embodiment of the invention to use known methods, such as TILLING (Targeting Induced Local Lesions IN Genomics; McCallum et al., 2000, Nat Biotech 18:455, and McCallum et al. 2000, Plant Physiol. 123, 439-442) and EcoTILLING, to induce or identify mutations in CHR12 alleles and/or CHR12 promoters and to use the identified mutations to generate plant lines which produce either lower levels or higher levels of one or more CHR12 proteins and/or chr12 mRNA transcripts according to the invention.

TILLING can be used generate (induce) and identify mutant plants or tissues comprising mutations in the CHR12 allele(s) which lead to a reduction in functional CHR12 protein being produced from such allele(s). Without limiting the scope of the invention, it is believed that mutations leading to higher levels of CHR12 protein could comprise point/deletion mutations in the promoter that are binding sites for repressor proteins that would make the CHR12 gene constitutive or higher in expression.

TILLING uses traditional chemical mutagenesis (e.g. EMS mutagenesis) followed by high-throughput screening for mutations in specific target genes (e.g. using Cel 1 cleavage of mutant-wildtype DNA heteroduplexes and detection using a sequencing gel system).

The method comprises in one embodiment the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such mutant plants. Seeds may for example be radiated or chemically treated and the plants screened for modified chromatin remodelling phenotype(s).

In another embodiment of the invention, the plant materials are natural populations of the species or related species that comprise polymorphisms or variations in DNA sequence at the CHR12 orthologous coding and/or regulatory sequence. Mutations at the CHR12 gene target can be screened for using a ECOTILLING approach (Henikoff S, Till B J, Comai L., Plant Physiol. 2004 June; 135(2):630-6. Epub 2004 May 21). In this method natural polymorphisms in breeding lines or related species are screened for by the above described TILLING methodology, in which individual or pools of plants are used for PCR amplification of the CHR12 target, heteroduplex formation and high-throughput analysis. This can be followed up by selecting of individual plants having the required mutation that can be used subsequently in a breeding program to incorporate the desired CHR12-orthologous allele to develop the cultivar with desired trait.

Thus, in one embodiment non-transgenic mutant plants which produce lower levels or higher levels of CHR12 protein and/or mRNA in one or more tissues are provided, or which completely lack CHR12 protein in specific tissues or which produce a non-functional CHR12 protein in certain tissues, e.g. due to mutations in one or more endogenous CHR12 alleles. For this purpose also methods such as TILLING and/or EcoTILLING may be used. Seeds may be mutagenized using e.g. radiation or chemical mutagenesis and mutants may be identified by detection of DNA polymorphisms using for example CEL 1 cleavage. Non-functional CHR12 alleles may be isolated and sequenced or may be transferred to other plants by breeding methods.

Mutant plants can be distinguished from non-mutants by molecular methods, such as the mutation(s) present in the DNA, CHR12 protein levels, CHR12 RNA levels etc, and by the modified phenotypic characteristics.

The non-transgenic mutants may be homozygous or heterozygous for the mutation conferring the enhanced expression of the endogenous CHR12 gene(s) or for the mutant CHR12 allele(s).

### Uses According to the Invention

Also various uses of the CHR12 nucleic acid and CHR12 amino acid sequences are provided. Similarly, various uses for the CHR12 promoters are provided.

In one embodiment the use of a nucleic acid sequence encoding a chromatin remodeling protein for the generation of transgenic plants or plant parts having modified growth characteristics is provided, characterized in that the nucleic acid sequence is selected from the group consisting of:

(a) a nucleic acid sequence encoding a protein of SEQ ID NO: 1;
(b) a nucleic acid sequence encoding a protein having at least 70% amino acid identity to SEQ ID NO: 1 over the entire length;
(c) a (sense and/or antisense) fragment of at least 15 consecutive nucleotides of the sequence of (a) or (b).

Preferably the modified growth characteristics are one or more of the group consisting of:

(a) biotic and/or abiotic stress-dependent growth arrest of one or more tissues, such as the primary stalk or inflorescences;
(b) biotic and/or abiotic stress-dependent dormancy-like growth arrest of underground storage organs (e.g. of tubers, bulbs, etc.);
(c) biotic and/or abiotic stress-dependent delayed/prevented bolting (e.g. of leaf vegetables).

### Screening Methods According to the Invention

In addition a method for identifying genes involved in plant growth arrest or dormancy or for verifying gene function is provided. The method comprises the steps of:

(a) generating a transgenic plant or plant part which express the protein of SEQ ID NO: 1 or a protein comprising at least 50, 60, 70% (or more) amino acid identity to SEQ ID NO: 1 over the entire length; and
(b) identifying genes (or gene transcripts) which are differentially expressed in one or more tissues of the transgenic plants of (a) compared to non-transgenic controls or empty-vector controls.

The transgenic plant or plant tissue can be generated as described above. It is not necessary to generate stable transformants, although this is preferred. For differential expression analysis any known method may be used, such as cDNA-AFLP, differential hybridization and the like. Genes which are upregulated in the transgenic tissue can then be cloned and sequenced and their function in growth retardation/arrest or dormancy can be verified by making transgenic plants.

### Sequences

SEQ ID NO 1: amino acid sequence of the ATCHR12 protein from Arabidopsis thaliana (AtCHR12). Amino acid 406 to 695 depicts a SNF2 domain and amino acid 748 to 827 depicts a Helicase-C domain.

SEQ ID NO 2: cDNA of the AtCHR12 gene.

SEQ ID NO 3: ORF of the AtCHR12 gene.

SEQ ID NO 4: genomic sequence of the AtCHR12 gene of ecotype Columbia.

SEQ ID NO 5: promoter sequence of the AtCHR12 gene.

### FIGURE LEGENDS

FIG. 1. Activation-tagged mutant of AtCHR12 gene

(A) A chromosomal position (bp) of the activating I element carrying a tetramer of the CaMV 35S enhancer and BAR gene in AtCHR12ov mutant.

(B) Semi-quantitative RT-PCR using AtCHR12 (top) or actin (bottom) primers.

(C) Primary inflorescence of Ws wild type.

(D-F) The growth arrest of primary inflorescence of AtCHR12ov mutant. Arrow (F) indicates primary inflorescence overgrown by lateral shoots.

FIG. 2. The effect of water deprivation on AtCHR12 mutants

(A) Primary inflorescence of Ws wild type and AtCHR12ov mutant after 10 days of water deprivation.

(B) Reduced growth of primary stem of four independent AtCHR12_tov lines. Stem height after 7 days of water deprivation is presented as percentage relative to Ws wild type plants. Errors bars represent SEM (n=10).

(C) The growth of primary inflorescence of atchr12 knockout mutant compares to Col wild type plants after 5 days of water deprivation.

FIG. 3. The effect of heat on AtCHR12 mutants

(A) The development of primary inflorescence of Ws wild type and AtCHR12ov mutant following heat stress at 37° C. for 24 h and 5 days of recovery at 22° C.

(B) Root elongation after 5 days of recovery at 22° C. after the heat stress treatments. Root length is presented as percentage relative to length of untreated controls. Error bars represent SEM (n=30).

(C) Seedlings survival after heat shock at 45° C. and 7 days of recovery at 22° C.

FIG. 4. Inhibition of root growth by salt is impaired in atchr12 mutant

(A) Root elongation after 5 days of growth on media with indicated NaCl concentration of Ws wild type and AtCHR12ov mutant.

(B) Root elongation of Col wild type and atchr12 knockout mutant.

Root length is presented as percentage relative to elongation on medium without salt.

Error bars represent SEM (n=30).

FIG. 5. Histochemical analysis of GUS reporter expression from AtCHR12 promoter

(A) Embryo showing GUS positive radicle.

(B) Strong GUS activity in radicle of dry seed.

(C) 1-day old seedling with GUS activity in cotyledon and upper hypocotyl.

(D) 3-day old seedling with diminishing GUS activity in expanding cotyledons.

(E) GUS activity in endodermis of root elongation zone of 3-days old seedling.

(F) 3-day old seedling without detectable GUS activity in growing shoot meristem.

(G) Young axillary bud.

(H) Opened young axillary bud.

(I) Primary bud.

(J) Arrow pointing to stipule, shown in detail in (K).

Bars=50 μm in (A), (B), (E), (F) and (K), and 1 mm in (C), (D), (G), H), (I) and (J).

FIG. 6. Valorisation of microarray data

Semi-quantitative RT-PCR analysis of genes identified as differentially expressed in AtCHR12ov mutant in primary inflorescence of 4 weeks old plants. As quantitative control a ubiquitin primers were used.

FIG. 7. Expression analysis of AtDRM1s genes in AtCHR12 mutants

(A) Semi-quantitative RT-PCR analysis of primary and axillary buds 5 days after bolting on Ws wild type and AtCHR12ov mutant.

(B) Analysis of primary inflorescence of 4 weeks old plants of both AtCHR12 mutants and their corresponding wild types.

(C) Analysis of rosette leaves of 4 weeks old plants of both AtCHR12 mutants and their corresponding wild types.

Experiments were repeated twice and similar results were obtained. As quantitative control a ubiquitin primers were used.

FIG. 8. The effect of water withholding on AtCHR12 mutants.

(a) Increase in length of the primary stem of wild-type and mutant plants after 6 days of water withholding.

(b) Increase in length of the primary stem of wild-type and mutant plants grown at standard conditions. Errors bars represent 2×SE. Asterisks indicate significant differences in the response of mutants relative to their corresponding wild-type. *, P<0.05; **, P<0.01; *** P<0.001.

FIG. 9. The effect of heat stress on AtCHR12 mutants.

(a) Increase in the length of the primary stem after heat stress of 37° C. for 16 h in wild-type (Ws) and AtCHR12ov plants. Control, non-treated, plants were grown and measured in parallel with stressed plants. The elongation period was defined as the number of days from the start of the temperature treatment (day zero).

(b) Increase in the length of the primary stem after heat stress of 37° C. for 16 h in wild-type (Col) and atchr12 mutant plants. Control, non-treated, plants were grown and measured in parallel with stressed plants. The elongation period was defined as in (a).
(c) Root length of wild-type (Col) and atchr12 seedlings after 5 days of recovery at 22° C. after the heat stress treatments. Control roots were grown in parallel at 22° C. Error bars represent 2×SE. Asterisks indicate significant differences in response of mutants relative to their corresponding wild-type. *, P<0.05; **, P<0.01; *** P<0.001.

FIG. 10. The influence of salt on root growth.

(a) Root length after 5 days of growth on media with different concentrations of NaCl in wild-type (Ws) and the AtCHR12ov mutant.

(b) Root length of wild-type (Col) and atchr12 knockout mutant as in (a).

Error bars represent 2×SE. Summary statistics is given in Supplementary Table S2. Asterisks indicate significant differences between the response of mutant and wild-type plants, at given NaCl concentration. **, P<0.01; *** P<0.001.

FIG. 11.

The effect of temperature on germination of freshly harvested seeds of wild type (Ws) and AtCHR12ov over-expressing mutant. Seeds were sown on filter paper moistened with water. Germination was scored after 4 days. Values are the mean±SD of three replicates.

FIG. 12.

The effect of after-ripening at room temperature on dormancy breaking and germination. Seeds were sown on filter paper moistened with water. Germination was scored after 4 days incubation at 20° C. Values are the mean±SD of three replicates.

FIG. 13.

Induction of secondary dormancy in 2 months old after-ripened seeds by incubation for 10 days in dark at 20° C. To break induced SD-dormancy (secondary dormancy) the seeds were subsequently stratified for 2 days at 4° C. Control seeds were germinated in the light at 20° C. Values are the mean±SD of three replicates.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

### EXAMPLES

### Example 1

### Identification of an Activation-Tagged Mutant Over-Expressing AtCHR12

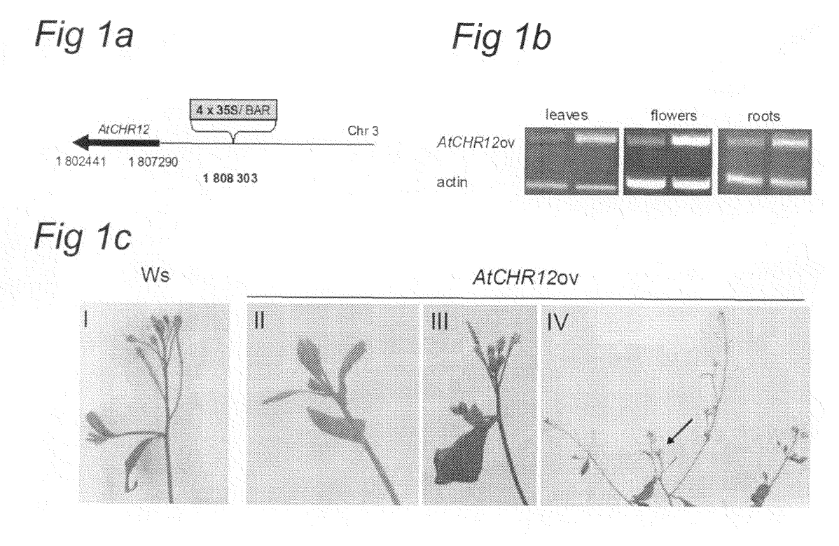

Activation-tagged mutant of AtCHR12 was identified in a population of activation-tagged lines in ecotype Wassilijevskaja (Ws) carrying the En-I maize transposon system with four tandem copies of the 35S enhancer sequence (Marsch-Martinez, et al., 2002, Plant Physiol 129, 1544-1556). From a population of about 1700 single-copy stable insertion lines (Dr. A. Pereira, PRI, Wageningen, unpublished), the flanking sequences were blasted against the genomic sequences of all known or predicted chromatin remodeling genes (from http://www.chromdb.org). In each case, the transcribed region and 10 kb of upstream and downstream surrounding sequences were included in Blast analysis. Through this screen, insertion line with enhancer sequence integrated about 1 kb upstream of the transcription initiation site of AtCHR12 (At3g06010) (FIG. 1A). The integration site was confirmed by PCR (data not shown). Over-expression of AtCHR12 in this line was examined by semi-quantitative RT-PCR analysis, showing a considerable up-regulation of the expression of this gene in leaves, flowers and roots (FIG. 1B). To compare the behavior of the over-expressed allele with a loss-of-function allele of the same gene, a SALK T-DNA line (SALK_{—}105458) in ecotype Columbia with the T-DNA insertion in the first exon of AtCHR12 (Alonso, 2003, Science 301, 653-657) was obtained and analyzed in parallel. In the remainder, the over-expressed allele of AtCHR12 will be indicated with AtCHR12ov and the knockout with archr12

### Example 2

### Temporary Growth Arrest of the Primary Inflorescence in AtCHR12ov

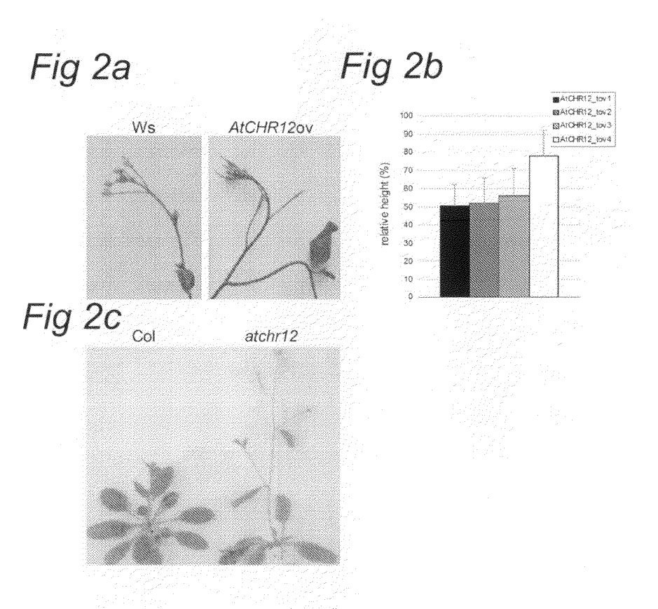

The phenotype of the over-expression of AtCHR12 with respect to plant development was studied in homozygous plants of the F3 generation. Four weeks after germination, mutant plants were indistinguishable from the wildtype (ecotype Ws). Neither bolting of the inflorescence, nor the initiation of lateral branches appeared affected. However, after the primary inflorescence stem had reached a length of about 8-10 cm, growth arrest of the primary inflorescence was observed in 10-20% of the mutant plants compared to the wildtype (FIG. 1C). The primary inflorescence, possibly but not necessarily carrying a few open flowers or developing siliques, stopped to grow (FIG. 1D-F). In approximately half of the plants with this phenotype, the primary inflorescence remained arrested for the rest of the life of the plant (FIG. 1E). Because the growth of lateral shoots from the primary stem and axillary shoots from the rosette was not affected, the main shoot became overgrown (FIG. 2F). In other plants with the growth-arrested phenotype, the activity of the primary inflorescence reinitiated after 1-2 weeks, resulting in numerous new flowers. This indicate that at optimal growth conditions, the growth arrest of the primary inflorescence is a very subtle phenotype. AtCHR12ov plants were normally fertile and did not show any other obvious morphological or developmental differences relative to the wildtype (data not shown). Also, the loss-of-function SALK T-DNA insertion line, atchr12, did not show any visible phenotypic difference compared to its wild type (ecotype Columbia; data not shown) when grown under the same conditions.

To confirm in an independent way that the growth-arrest phenotype in the AtCHR12ov mutant is the result of AtCHR12 gene activation, transgenic plants with over-expressed AtCHR12 were generated and analyzed. The genomic sequence of AtCHR12 was isolated from ecotype Ws by PCR, placed under the control of the potato Lhca3.St.1 promoter (Nap, 1993, PMB 23, 605-612) and transformed into the wild type (ecotype Ws). The growth-arrest phenotype was recovered in several of the transgenic lines (data not shown). Individual transformants showed growth arrest of the primary inflorescence at different levels, probably reflecting various levels of transgene expression and position effects (Mlynarova, 1994, Plant Cell 6, 417-426). In the remainder of the Examples the over-expressed transgenic alleles of AtCHR12 will be designated AtCHR12_tov.

### Example 3

### AtCHR12 is Involved in Responses to Environmental Stress

Growth of plants is arrested at various abiotic stresses (Zhu, 1997 Plant Sciences 16, 253-277; Smallwood, 1999 “Plant responses to environmental stress”, Oxford UK: BIOS Scientific Publishers; Shinozaki, 2000, Curr Opin Plant Biol 3, 217-223). To investigate if such a response to adverse environments is altered in the AtCHR12 mutants, we challenged AtCHR12ov and atchr12 plants with 3 environmental stresses (drought, heat and salinity) and compared the reaction to their corresponding wild type. In addition, we also evaluated the effect of ABA on AtCHR12-related growth arrest.

The effect of drought was analyzed by growing plants for 4-5 weeks under standard conditions, followed by a period 10-14 days of suspended water supply before starting re-watering. During this period, the timing and development of the primary inflorescence was studied. In wild type (ecotype Ws) plants, the primary inflorescence appeared normal, exhibiting only moderately delayed development with respect to the untreated control. Growth arrest of the primary inflorescence was observed in 70-80% of the AtCHR12ov plants, compared to 10-20% among non-treated plants. FIG. 2A shows the primary inflorescence of wild type and AtCHR12ov plants after 10 days of exposure to drought stress. The same phenotype was observed in several of the transgenic over-expressing AtCHR12_tov lines. These overexpressing plants also showed considerably reduced growth of the stem. The length of their primary stem was reduced to about 50-70% of the wild type (ecotype Ws) (FIG. 2B). Upon relieving drought stress, most plants (both AtCHR12ov and AtCHR12_tov) resumed normal growth. Probably as a consequence of the growth arrest, mutant plants ceased flowering 10-12 days later than the corresponding wild type plants. In contrast, whereas wild type (ecotype Col) showed stress-induced growth delay (FIG. 2C), the growth of the primary inflorescence of knockout atchr12 plants upon 5 days of water deprivation was indistinguishable from the untreated control (FIG. 2A). However, when water stress was prolonged to 2 weeks atchr12 plants somehow dried out faster than wild Col plants (data not shown).

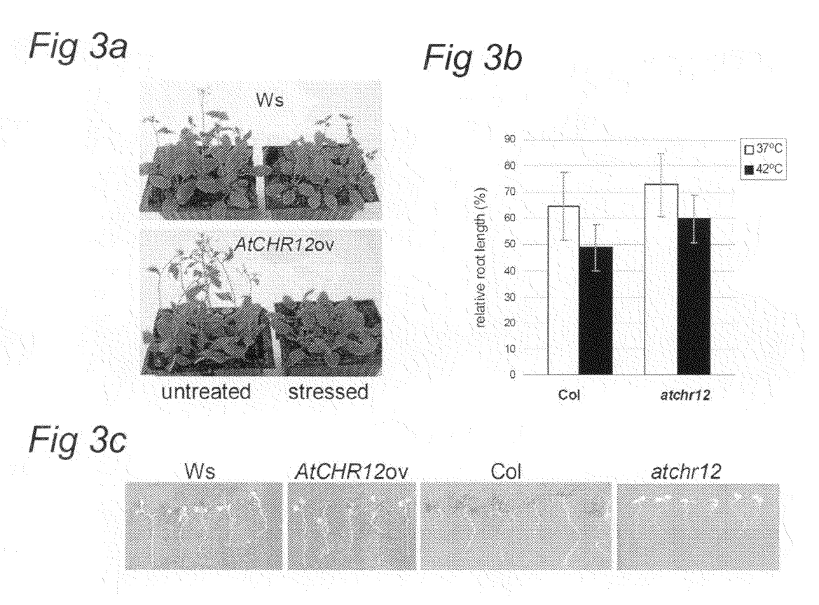

The influence of heat stress on four-week-old plants shortly after bolting was studied by exposing the plants to 37° C. for 16 or 24 h at dim light conditions and returning them to 22° C. for recovery. The effect on the development of flowers was assessed daily. After five days of recovery, all wild type plants (ecotype Ws) appeared normal, exhibiting only a moderate delay in growth compared to non-treated controls (FIG. 3A). In contrast, the growth of AtCHR12ov plants was severely affected. In plants stressed for 24 h, already one day after the heat stress all primary inflorescences wilted and died (FIG. 3A). Newly formed axillary shoots developed somewhat later compared to wild type, but had normal morphology. Plants exposed to stress for 16 h showed similar growth arrest of the primary inflorescence as observed for drought-stressed plants (data not shown). This indicates that the strength of growth arrest response is modulated depending on the severity of the stress. The atchr12 plants did not show any difference in response after exposure to the same treatment compared to their wild type (ecotype Col; data not shown).

In view of these results, we decided to investigate the heat response in other growth stages of the various plant lines. Three-day-old in vitro seedlings were exposed for 5 h to 37° C. or 42° C. Following heat treatment, seedlings were allowed to recover for 5 days at 22° C., then the length of the roots was measured and compared to untreated controls. Both temperatures had a negative effect on root growth (FIG. 3B), but the growth of atchr12 roots was less inhibited than in its corresponding wild type (ecotype Col; FIG. 3B). AtCHR12ov mutant did not differ from ecotype Ws (FIG. 3B). In addition, five-day-old seedlings were challenged for survival by a heat shock of 45° C. for 1.5 hour. Under this condition, none of the knockout seedlings survived, in contrast to over 90% of the wild type seedlings (ecotype Col). The survival of both Ws and AtCHR12ov seedlings was about 50% (FIG. 3C).

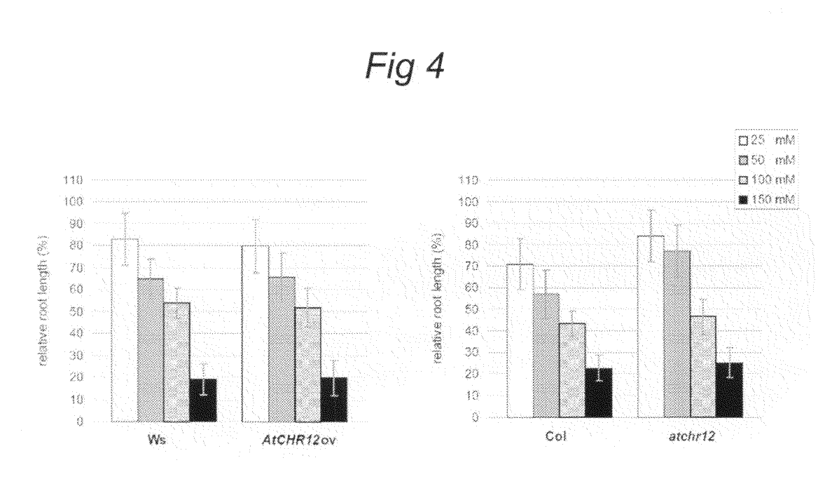

The responses of both AtCHR12 mutants to high salt stress were investigated with the help of an in vitro root elongation assay (Achard, 2006 Science 311, 91-94). Three-day-old seedlings were transferred to agar plates containing a range of 25-150 mM NaCl, and root length was measured after 5 days of incubation. There was no difference in root length of Ws and AtCHR12ov seedlings in any of the salt concentrations analyzed (FIG. 4A). However, the growth of atchr12 roots was less inhibited by salt than in its corresponding wild type (ecotype Col), especially at lower salt concentrations (FIG. 4B). Growing the seedlings of both mutants in the presence of 1 μM ABA did not reveal any difference in inhibiting effect of ABA on root growth (data not shown). Mutant plants grown in vitro on medium with 1 μM ABA did not show any difference in flowering time with respect to their wild type controls as well (data not shown). These data combined indicate that AtCHR12 is involved in stress-induced growth arrest. The over-expressors, both AtCHR12ov and AtCHR12_tov, show increased growth arrest. In contrast, the atchr12 knockout shows less growth arrest when challenged by relatively mild stress conditions.

### Example 4

### The AtCHR12 Promoter GUS Fusion is Active in Tissues with Growth Arrest

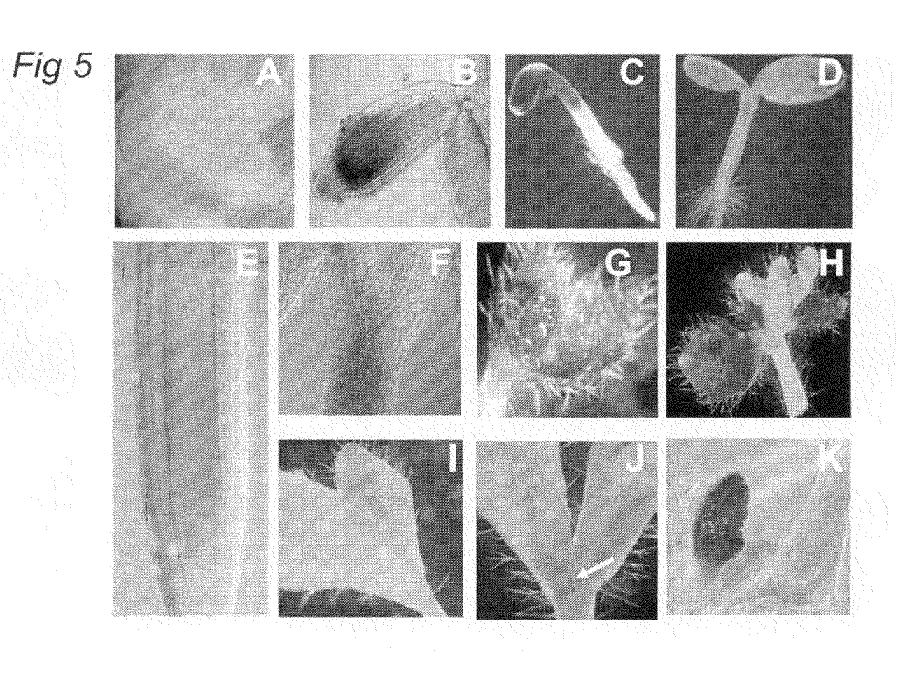

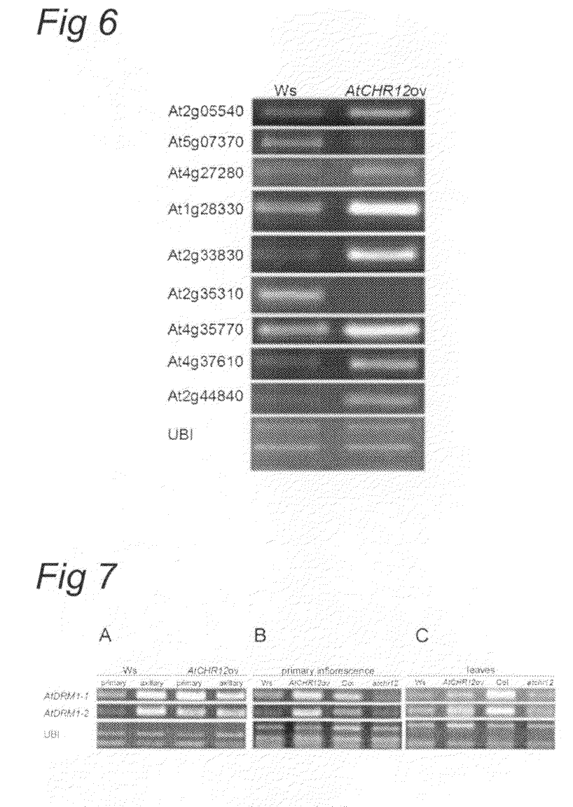

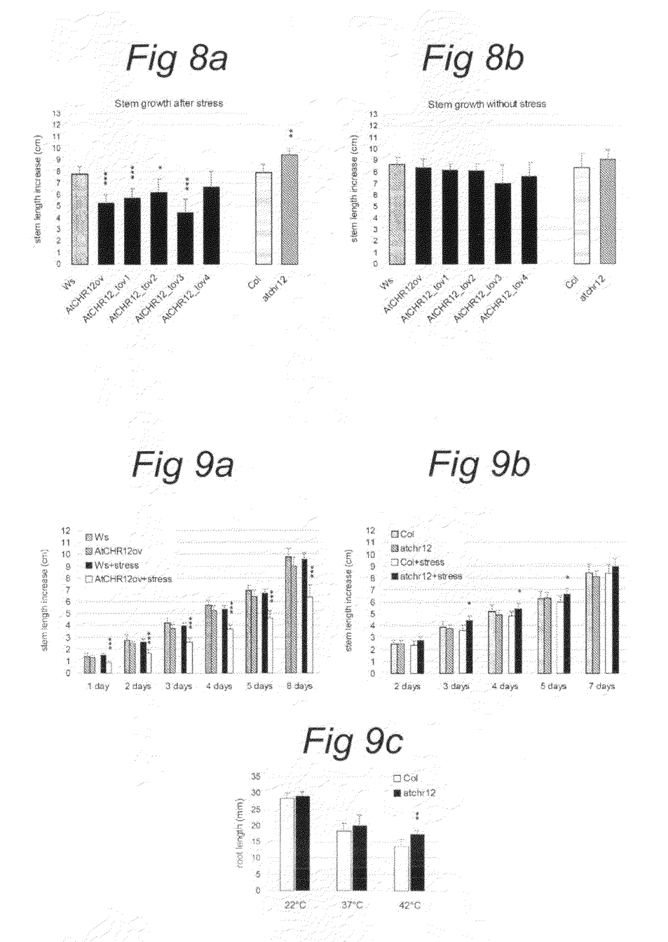

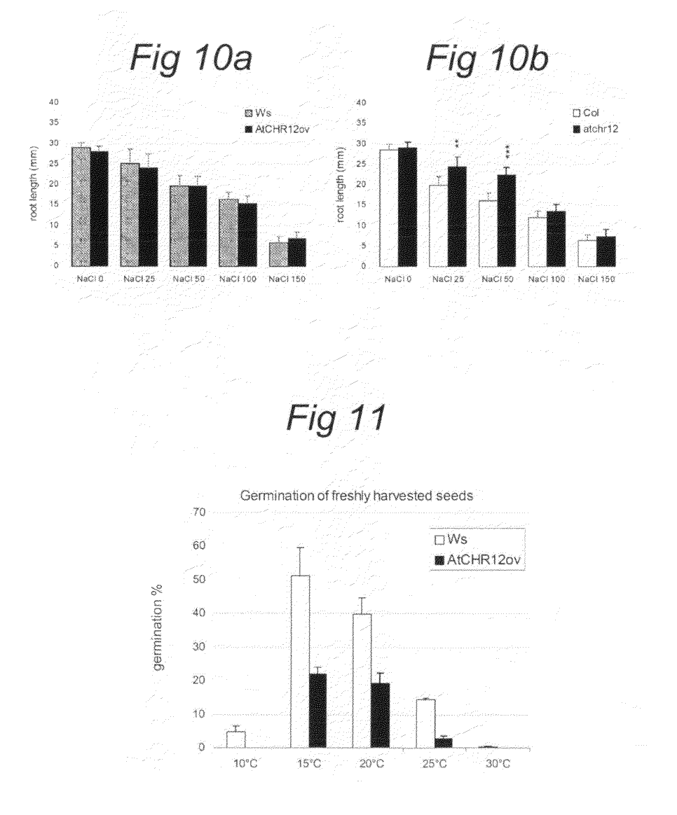

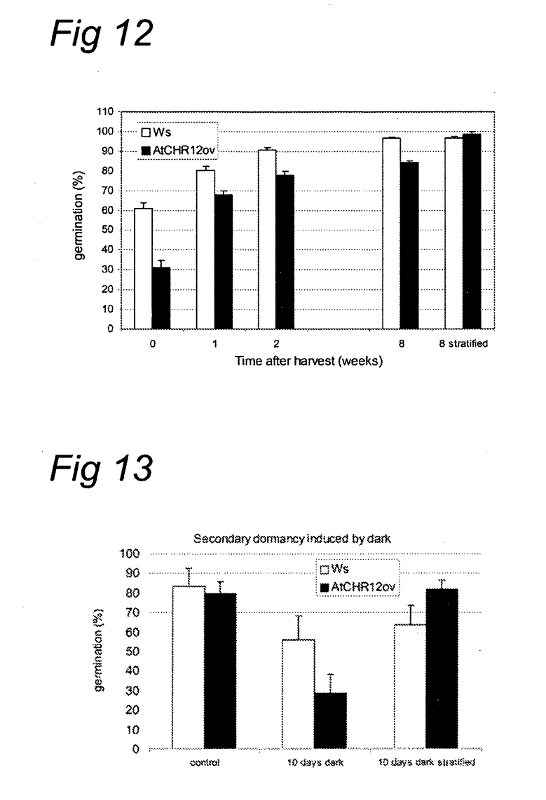

To characterize the spatial and temporal expression of the AtCHR12 gene, transgenic plants (ecotype Ws) harbouring a chimeric construct of the AtCHR12 promoter (1.5 kb, SEQ ID NO: 5) fused to gus (construct designated pCHR12::GUS) were generated by Agrobacterium tumefaciens-mediated transformation. GUS activity was detected in the hypocotyl of the embryo from the mid-torpedo stage on (FIG. 5A) and in dry seeds after 1 h of imbibition (FIG. 5B). In developing seedlings, one day after germination, strong GUS activity was observed in cotyledons and upper hypocotyls (FIG. 5C). When the seedlings became older, the GUS activity in expanding cotyledons and hypocotyls declined (FIG. 5D). In the division zone of the root endodermis (6-8 cells above the quiescent centre), GUS activity was first observed in three-day-old seedlings and remained detectable during further development (FIG. 5E). GUS activity could not be detected in the shoot meristem (FIG. 5F). In plants growing in soil, intense GUS staining was present in young axillary rosette buds and lateral buds developing from the main stem (FIG. 5G). The high GUS activity was localized mainly in developing cauline leaves in the early stages of their development, when they were enclosing the emerging inflorescences (FIG. 5H). In older cauline leaves GUS activity diminished. The primary buds were free of GUS activity (FIG. 5I), while strong GUS staining was observed in both rosette and cauline leaf stipules (FIG. 5J, K).

### Example 5

### Microarray Analysis Correlates the Expression of AtCHR12 with Dormancy-Associated Genes

To get more insight into the mode of action of AtCHR12 and to identify possible downstream target genes, microarray analysis was carried out using Agilent 44K Arabidopsis 3 oligo arrays. RNA from the primary inflorescence (including shoot and floral meristems) from four-week-old AtCHR12ov plants, just before the visible phenotype, was compared with RNA from the corresponding tissue from the wild type (ecotype Ws). From 482 genes that were differentially expressed (at p<0.001), more than a 2-fold change in expression was observed for only 38 up-regulated (Table 1) and 30 down-regulated (Table 2) genes. The most up-regulated gene was AtCHR12 (11-fold), which can be taken as internal control for the quality of the array analysis. In each of the nine case analyzed, RT-PCR confirmed the differential expression observed in the microarray analysis (FIG. 6). For most genes, probe hybridization in RNA blots was considered inappropriate to achieve independent confirmation of the microarray results, because they showed low expression or their probes could potentially cross hybridize with other genes.

TABLE 1
Genes showing at least 2-fold up-regulated in AtCHR12ov mutant

| TAIR | | Fold | |
|---|---|---|---|
| Annotation^{a} | Sequence description | change | p value |
| At3g06010 | homeotic gene regulator | 11.3 | 1.34e−29 |
| At4g35770 | senescence-associated protein (SEN1) | 5.76 | 8.52e−29 |
| At2g33830 | dormancy/auxin associated protein (AtDRM1-2) | 4.67 | 1.05e−29 |
| At4g27280 | calcium-binding EF hand family protein | 3.69 | 6.22e−12 |
| At2g05540 | glycine-rich protein | 3.57 | 2.34e−12 |
| At3g44260 | CCR4-NOT transcription complex protein | 3.46 | 5.38e−13 |
| At4g24570 | mitochondrial carrier protein | 3.46 | 1.07e−15 |
| At4g17340 | major intrinsic family protein | 3.08 | 6.16e−04 |
| At1g28330 | dormancy/auxin associated protein (AtDRM1-1) | 3.04 | 9.19e−08 |
| At4g37610 | TAZ zinc finger family protein | 2.78 | 4.13e−33 |
| At4g36740 | homeobox-leucine zipper family protein | 2.76 | 1.22e−05 |
| At1g07135 | glycine-rich protein | 2.68 | 1.79e−09 |
| At3g57520 | raffinose synthase | 2.64 | 0 |
| At5g20250 | raffinose synthase family protein | 2.57 | 6.26e−10 |
| At2g44840 | ethylene-responsive element-binding protein | 2.55 | 9.10e−04 |
| At2g22990 | sinapoylglucose:malate sinapoyltransferase | 2.45 | 4.74e−10 |
| At4g32020 | expressed protein NuLL | 2.34 | 7.84e−05 |
| At3g30775 | osmotic stress-induced proline dehydrogenase | 2.29 | 6.06e−10 |
| At1g31820 | amino acid permease family protein | 2.23 | 5.49e−04 |
| At3g15630 | expressed protein | 2.2 | 0 |
| At5g22920 | zinc finger (C3HC4-type RING finger) | 2.2 | 1.19e−24 |
| At2g05380 | glycine-rich protein | 2.14 | 3.12e−06 |
| At1g80920 | DNAJ heat shock domain-containing protein | 2.14 | 5.17e−08 |
| At2g40000 | expressed protein | 2.13 | 2.78e−06 |
| At4g34670 | 40S ribosomal protein S3A | 2.11 | 3.07e−09 |
| At1g76600 | expressed protein | 2.1 | 4.64e−11 |
| At5g28750 | thylakoid assembly protein | 2.1 | 1.08e−06 |
| At1g32920 | expressed protein | 2.08 | 8.72e−27 |
| At1g73540 | MutT/nudix family protein | 2.07 | 1.09e−09 |
| At3g15450 | similar to auxin down-regulated protein ARG10 | 2.06 | 4.03e−04 |
| At4g35060 | heavy-metal-associated domain-containing protein | 2.05 | 0 |
| At5g57560 | xyloglucan:xyloglucosyl transferase | 2.04 | 4.36e−07 |
| At3g47340 | asparagine synthetase 1 | 2.03 | 4.64e−10 |
| At5g50260 | cysteine proteinase | 2.03 | 8.65e−04 |
| At3g50060 | myb family transcription factor | 2.03 | 6.06e−16 |
| At2g18730 | diacylglycerol kinase | 2.03 | 9.85e−04 |
| At3g48740 | nodulin MtN3 family protein | 2.03 | 1.30e−04 |
| At1g18300 | MutT/nudix family protein | 2.02 | 9.20e−05 |

TABLE 2
Genes showing at least 2-fold down-regulation in AtCHR12ov mutant

| TAIR | | Fold | |
|---|---|---|---|
| Annotation | Sequence description | change | p value |
| At5g07370 | inositol polyphosphate 6-/3-/5-kinase (AtIPK2α) | 8.48 | 3.99e−08 |
| At2g35270 | DNA-binding protein | 7.73 | 7.34e−07 |
| At3g04370 | hypothetical protein | 6.63 | 1.46e−08 |
| At1g67090 | RuBisCO small subunit 1A | 3.52 | 7.73e−18 |
| At2g35290 | expressed protein | 3.5 | 2.94e−15 |
| At5g07230 | protease inhibitor/seed storage/lipid transfer protein | 3.13 | 4.41e−22 |
| At4g34850 | chalcone and stilbene synthase family protein | 3.08 | 4.51e−25 |
| At1g69940 | pectinesterase family | 2.99 | 4.74e−16 |
| At2g28355 | expressed protein | 2.98 | 2.62e−14 |
| At5g44540 | tapetum-specific protein-related | 2.95 | 5.39e−12 |
| At3g13220 | ABC transporter family protein | 2.87 | 2.97e−05 |
| At1g54040 | kelch repeat-containing protein | 2.78 | 4.95e−14 |
| At3g28780 | glycine-rich protein | 2.63 | 2.04e−08 |
| At2g35310 | transcriptional factor B3 family protein | 2.6 | 0 |
| At4g11760 | expressed protein | 2.58 | 2.96e−06 |
| At5g53820 | expressed protein similar to ABA-inducible protein | 2.57 | 4.94e−18 |
| At1g01280 | cytochrome P450 family protein | 2.54 | 1.98e−06 |
| At3g42960 | alcohol dehydrogenase (ATA1) | 2.53 | 5.85e−06 |
| At1g62940 | 4-coumarate-CoA ligase family protein | 2.48 | 1.26e−13 |
| At5g38410 | RuBisCO small subunit 3B | 2.43 | 9.54e−12 |
| At5g26730 | expressed protein | 2.4 | 3.04e−07 |
| At1g02930 | glutathione S-transferase | 2.31 | 8.21e−04 |
| At3g07450 | protease inhibitor/seed storage/lipid transfer protein | 2.3 | 3.23e−07 |
| At4g20050 | expressed protein | 2.24 | 8.22e−07 |
| At5g62080 | protease inhibitor/seed storage/lipid transfer protein | 2.22 | 8.25e−15 |
| At1g47980 | expressed protein | 2.2 | 2.25e−13 |
| At2g25510 | expressed protein | 2.13 | 6.92e−04 |
| At4g33355 | protease inhibitor/seed storage/lipid transfer protein | 2.1 | 3.02e−07 |
| At1g02050 | chalcone and stilbene synthase family protein | 2.06 | 6.97e−07 |
| At1g30795 | hydroxyproline-rich glycoprotein family protein | 2.04 | 1.19e−04 |

TAIR = www.arabidopsis.org

Two differentially expressed genes identified on the basis of microarray analysis could be easily associated with the growth-arrest phenotype: dormancy/auxin-associated genes. These genes, AtDRM1-1 (At1g28330) and AtDRM1-2 (At2g33830), show a 3-fold and 4.6-fold up-regulation, respectively. For these two genes, At1g28330 and At2g33830, probe hybridization was possible and the differential expression was also confirmed by RNA blot analysis (data not shown). These genes are the Arabidopsis orthologs of the pea dormancy-associated gene PsDRM1 (Stafstrom, 1997 Plant Physiol 114, 1632-1632). They have been shown to be repressed in growing organs and to be relatively highly active in dormant buds (Tatematsu, 2005 Plant Physiol 138, 757-766). To relate their expression characteristics to AtCHR12, the expression of these two genes were analyzed in both AtCHR12ov and atchr12 plants. Low expression of both AtDRM1s was observed in growing primary buds and high expression in axillary buds of wild type (FIG. 7A). The expression of both genes in AtCHR12ov was more pronounced in the primary buds compared to axillary buds or leaves (FIG. 7A, C), indicating that high expression of AtDRM1s in the primary inflorescence of ATCHR12ov could be associated with the transformation of an active inflorescence into a dormant inflorescence. The expression level of the AtDRM1s differs in the two ecotypes used (Ws and Columbia). Higher levels of expression were seen in ecotype Columbia in both inflorescence and leaves (FIG. 7B, C). In contrast, atchr12 showed reduced expression levels of both genes in both inflorescences and leaves compare to wild type Columbia (FIG. 7B, C). This confirms that the expression level of AtCHR12 is related to the processes in plants that are turning actively growing tissue into dormant-like tissue.

### Conclusions

The above experiments show that that Arabidopsis SNF2/Brahma-type ATPase gene AtCHR12 plays a role in regulating the genes conferring growth arrest notably upon the perception of stress. Modulation in AtCHR12 expression correlates with changes in expression of dormancy-associated genes and the ATCH12 protein is likely to be involved in establishing dormancy-like phenomena in plants. This establishes AtCHR12 as a novel gene involved in the response repertoire in plants to permits flexible modulation of plant growth in environment limitations.

### Example 6

### AtCHR12 Priming for Growth Arrest Differs from DELLA-Controlled Growth Restraint

DELLA proteins are major players in regulatory mechanisms for plant growth (Fleet and Sun, 2005, Curr. Opin. Plant Biol. 8, 77-85). They are thought to be nuclear transcriptional regulators that antagonize the growth enhancing effect of gibberellins (GAs). The phytohormone abscisic acid (ABA) counteracts the action of GAs and is a key player in plant responses to adverse environmental cues (Himmelbach et al., 2003, Curr. Opin. Plant Biol. 6, 470-479). Recently, the DELLA proteins were reported to be essential for induction of growth restraint upon salinity stress in an ABA-dependent manner. The growth of roots of an arabidopsis “quadruple-DELLA mutant”, with four out of five genes down-regulated, was less inhibited by salt stress than the growth of the root of wild-type plants (Achard et al., 2006, Science, 311, 91-94). This is similar to the response of the atchr12 mutant to salt stress, raising the possibility that DELLAs and ATCHR12 act in the same pathway.

However, seedlings of both types of AtCHR12 mutants grown in vitro on medium with 1 or 10 μM ABA did not reveal any differences in the effect of ABA on root growth or flowering time compared to their wild-type controls (data not shown). This was reported for DELLAs mutants (Achard et al., 2006, supra) and indicates that AtCHR12-associated growth arrest is ABA independent and differs from DELLA-mediated growth restraint. A DELLA-independent mechanism of growth arrest has been suggested to explain the incomplete resistance of DELLAs-mutant when salt stressed (Achard et al., 2006, supra). An interesting difference between DELLA-controlled growth restraint and AtCHR12-associated priming for growth arrest is that gain-of-function DELLA mutants show a constitutive dwarf phenotype caused by reduced GA responses (Fu et al., 2001, Plant Cell, 13, 1791-1802). In contrast, the growth restraint in the AtCHR12ov mutant and transgenic plants is stress dependent and reversible.

### Example 7

### AtCHR12 is Involved in Seed Dormancy

The inventors have shown that the AtCHR12 chromatin remodeling gene is active in developing and in dry seeds indicating the involvement in the embryo growth arrest during seed maturation or dormancy.

Two categories of seed dormancy are recognized. Primary dormancy (PD) which is acquired during seed development and refers to arrested germination of mature, fully imbibed seeds. Secondary dormancy (SD) generally occurs when dispersed, mature seeds are exposed for certain periods to environmental conditions that induce a quiescent state.

### AtCHR12 Overexpression Affects Primary Dormancy

With respect to PD, freshly harvested seeds of over-expressing mutant showed higher dormancy: lower percentage of germination (±50% of wild type) and more narrow temperature range of germination (FIG. 11).

The difference in germination between wild type and mutant was reduced by after-ripening at room temperature to a difference of only about ±12%. The lower germination percentages of mutant seeds were not due to seed mortality, because after cold stratification they germinated with the same frequency as wild type seeds (FIG. 12).

### AtCHR12 Overexpression Affects Secondary Dormancy

Secondary dormancy was induced in seeds that passed through primary dormancy (6 months old) by incubation for 10 days in dark. Moist incubation in dark at 20° C. reduced the ability to germinate subsequently in the light.

The secondary dormancy induced in mutant is stronger (lower % of germination) than in wild type seeds (FIG. 13). The ability of mutant SD seeds to germinate is fully regained after 2 days of dark stratification at 4° C., but not in wild type seeds. These data indicate the role of chromatin remodeling in control of dormancy cycling.

### Conclusions

Chromatin remodeling genes, such as AtCHR12 and variants and orthologs thereof, appear to be involved in seed germination and/or seed dormancy, both primary and secondary dormancy. The genes can, therefore, be used to generate plants and seeds for which dormancy maintenance and/or dormancy cycling can be controlled. For example, (over) expression of CHR12 genes in plants or in seeds (e.g. under a light-, temperature- or chemically inducible promoter) can be used to induce a stronger and/or more uniform seed dormancy and/or dormancy break and/or to control the length of the dormancy period. Vice versa, silencing of CHR12 genes or gene families can be used to reduce seed dormancy.

### Example 8

### Experimental Procedures

### Plant Material

An over-expressing AtCHR12ov mutant in the Wassilijewskaja (Ws) genetic background was identified in a population of activation tagged lines generated using the En-I maize transposon system described before (Marsch-Martinez et al., 2002, Plant Physiol. 129, 1544-1556). From a population of about 1700 stable, single-copy insertion lines (A. Pereira, unpublished), the flanking sequences were determined and blasted against the genomic sequences of all known or predicted chromatin remodeling genes (http://www.chromdb.org). In each case, the transcribed region and 10 kb of upstream and downstream sequences were included in the Blast analysis. The loss-of-function mutant atchr12, line SALK 105458 in the Columbia (Col-0) background, was generated by J. R. Ecker and the Salk Institute of Genomics Analysis Laboratory (USA) and distributed by NASC (Scholl et al., 2000 Plant Physiol. 124, 1477-1480). In this line, a single insertion is present. For both mutants, plants of the F3 generation homozygous for the insertion were used. Homozygosity was confirmed by plating seeds on plates with 15 mg/litre phosphinothricin-DL (AtCHR12ov) or 50 mg per litre kanamycin (atchr12). To synchronize germination, seeds were imbibed in distilled water for 3 days at 4° C.

### Stress Treatment

Stress treatments were conducted on plants grown either in soil or on solid MS medium. To induce drought stress, plants were grown in commercial compost comprised of 80% of a mixture of clay and turf and 20% perlite (Hortimea, Elst, The Netherlands) supplemented with Scotts Osmocote fertilizer. To avoid too rapid soil drying sufficiently large (9 cm diameter) and deep (9 cm) pots were used to grow 6 plants per pot. Pots with mutant and wild-types plants were placed next to each other on the same tray. After germination plants were grown in a growth chamber with 16/8 h light/dark cycle at 22° C., light intensity 100 μmol m⁻² s⁻¹ and relative humidity 57-80%. Plants were watered daily to the tray. Three-week-old (Ws background) or 4-week-old plants (Col-0 background) were submitted to progressive drought stress by withholding water supply.

For heat stress, 3-week-old (Ws background) or 4-week-old (Col-0 background) plants were placed for 16 h in a growth chamber at 37° C. and subsequently returned to 22° C. for recovery. For both treatments, the floral development was assessed visually; the stem length of individual plants was measured using a ruler as the distance from the base to the first flower on the stem. For root elongation assays, seeds were surface sterilized and plated on 0.8% w/v agar (Daishin; Duchefa, Haarlem, The Netherlands), 0.5×MS medium (Murashige and Skoog, Duchefa) supplemented with 1% w/v sucrose and 0.5 g 1-1 MES, pH 5.8. Following the cold treatment for 3 days at 4° C. in darkness, seedlings were grown in a controlled growth chamber with a 16/8 light/dark cycle at 22° C. in a vertical position. Three-day-old seedlings were transferred to plates supplemented with 0, 25, 50, 100 or 150 mM NaCl and grown in a vertical position. Five days later, the root length of 20-30 seedlings was measured. Heat treatments were performed on three-day-old seedlings growing on plates that were directly heated in an incubator at 37° C. or at 42° C. for 5 h. After recovery at 22° C. for 5 days, the length of the roots was measured and compared to untreated controls.

### PCR and RT-PCR Analyses

The T-DNA integration in the proximity of the AtCHR12 start of transcription in the AtCHR12ov mutant was confirmed by PCR using a genomic primer (5′-CCAAAGTGACATCTCATGG-3′, SEQ ID NO: 6) and a primer (5′-CTTACCTTTTTTCTTGTAGTG-3′, SEQ ID NO: 7) from the En-I element, originally used for sequencing plant flanking DNA (Marsch-Martinez et al., 2002, Plant Physiol. 129, 1544-1556). The T-DNA integration into the first exon of the AtCHR12 gene in the atchr12 mutant was confirmed using a gene specific primer (5′-GCCTCACCCTAGATTTTGATG-3′, SEQ ID NO: 8) and a primer (5′-GCGTGGACCGCTTGCTGCAACT-3′, SEQ ID NO: 9) from the left border of the T-DNA (LBb1). Methods for DNA isolation and RT-PCR conditions were described previously (Mlynarova and Nap, 2003, Transgenic Res. 12, 45-57; Mlynarova et al., 2003, Plant Cell, 15, 2203-2217). Two micrograms of total RNA was used to synthesize the first-strand cDNA using an oligo(dT) primer. The cDNA was diluted 50 times and first used for amplification using ubiquitin primers (32 cycles) to equalize the concentrations of the cDNA samples. Subsequently, appropriately diluted cDNA was used for PCR reactions (35 cycles) using gene-specific primers. Reactions for control and tested genes were preformed in parallel, but in separate tubes. For each gene, a series of diluted cDNA was taken and adjusted to ensure that the PCR product shown was generated in the exponential stage of amplification. Generally, the lowest amount of cDNA still giving an ethidium bromide stained product was taken to represent the RT-PCR. The products were visualized on 1.2-1.5% agarose gels. Sequences of primers used to confirm microarray data are given in SEQ ID NO: 10-31, with primer pairs as follows:

SEQ ID NO: 10 and 11—gene At2g05540
SEQ ID NO: 12 and 13—gene At5g07370
SEQ ID NO: 14 and 15—gene At4g27280
SEQ ID NO: 16 and 17—gene At1g28330
SEQ ID NO: 18 and 19—gene At2g33830
SEQ ID NO: 20 and 21—gene At4g35770
SEQ ID NO: 22 and 23—gene At2g35310
SEQ ID NO: 24 and 25—gene At4g37610
SEQ ID NO: 26 and 27—gene At3g44260
SEQ ID NO: 28 and 29—gene At2g44840
SEQ ID NO: 30 and 31—gene ubiquitin.

### Generation of AtCHR12 Toy Transgenic Plants

The sequence of the AtCHR12 gene (At3g06010; 4850 bp, including 11 introns; TAIR, http://www.arabidopsis.orgf) was obtained by amplification from genomic DNA from accession Ws using the Phusion™ DNA polymerase (Finnzymes, Finland). The full length sequence was obtained with 3 sets of primers: CHRI (SEQ ID NO: 32) for 5′-GGATCCTCATGAAGGCTCAGCAGCTCCAAGAG-3′ and CHRIrev (SEQ ID NO: 33) 5′-CCTTCTAATTGATAGGATCGTAG-3′ amplifying fragment I from sequence 1-2290 bp; CHRII (SEQ ID NO: 34) for 5′-GGCTATCCATTCAATACAAGAG-3′ and CHRIIrev (SEQ ID NO: 35) 5′-GGGTTCCAATCACTGTCAAG-3′ amplifying fragment II from sequence 2120-3888 bp; CHRIII (SEQ ID NO: 36) for 5′-CAATTCAACGAGCCAGATTCTC-3′ and CHRIIIrev (SEQ ID NO: 37) 5′-CTCGAGTCATTTTCGTCTACTTCCAT-3′ amplifying fragment III from sequence 3791-4850 bp. The BamHI and SstI sites (underlined) were introduced via the PCR primers for cloning purposes. All fragments were cloned into pGEM-Teasy (Promega) and their integrity was verified by sequencing. Next, the cloned fragments were assembled into the gene sequence: fragment I (BamHI-XbaI) was fused to fragment II (XbaI-PstI) and fragment III (PstI-SstI). Restriction XbaI site is present at position 2269 in AtCHR12 gene, PstI is a unique restriction site at position 3853. The full gene sequence was ligated to the potato Lhca3.St.1 promoter (Nap et al., 1993, Plant Mol. Biol. 23, 605-612). Using Gateway technology (Invitrogen), the whole cassette was introduced into the vector pBnRGW (unpublished). This binary vector consists of the backbone sequence of pB7GWIWG2(II) (http://www.psb.ugent.be/gateway/index.php) into which the napin promoter-DsRFP-nosT cassette from pFLUAR 101 (Stuitje et al., 2003, Plant Biotechnol. J. 1, 301-309), the Gateway exchange cassette and the nosT polyadenylation sequence were introduced by replacing the XbaI-HindIII T-DNA fragment with standard cloning. The final binary vector was introduced into Agrobacterium tumefaciens C58C1 (pMP9) and used for transformation of Arabidopsis thaliana (accession Ws) according to the floral dip method (Clough and Bent, 1998, Plant J. 16, 735-743).

AtCHR12 Promoter Fusion with Gus and Histochemical GUS Assay

The AtCHR12 promoter (1480 bp) was isolated with PCR from genomic DNA from accession Ws using the primers (SEQ ID NO: 38) 5′-GTTAGTGGAAGCCTTTATGAGCC-3′ and (SEQ ID NO: 39) 5′-GCCACCATGGCGGGAACTTG-3′. The PCR fragment was cloned into pGEM-Teasy, verified by sequencing and subsequently ligated to gus and nosT polyadenylation sequence. The pCHR12-gus-nosT cassette was cloned into a derivative of the binary vector pBinPLUS (van Engelen et al., 1995, Transgenic Res. 4, 288-290) containing a cassette of napin promoter-DsRFP-nosT for selection. The resulting binary plasmid was used for transformation as described above. Three independent transgenic lines were analyzed histochemically for GUS activity. Samples were vacuum infiltrated for 15 min in GUS staining buffer (Jefferson et al., 1987, EMBO J. 6, 3901-3907), consisting of 100 mM sodium phosphate, pH 7.0; 10 mM EDTA; 0.5 mM K₄Fe[Cn]₆; 0.1% w/v Triton X-100 and 1 mM X-gluc (Duchefa) and incubated for 6-18 h at 37° C. To ensure better penetration of the substrate in developing seeds, siliques were partially opened with a needle before vacuum infiltration. Dry seeds were imbibed for a few minutes in GUS buffer, peeled and further incubated at 37° C. overnight. GUS staining was observed with a Nikon SMZ-U zoom 1:10 binocular microscope or a Nikon Optihot-2 stereomicroscope, and recorded using a digital camera (Nikon coolpix 995). Images were processed with Paint Shop Pro9.

### Statistical Analysis

To test if the response of mutant plants is significantly different from their corresponding wild-type plants, a two-sample unequal variance t-test was used. In graphs, error bars are equal to 2× the standard error (SE). They are drawn on top of the mean values, that is approximately equivalent to the 95% confidence interval (Streiner, 1996). Asterisks indicate significant difference in the response of mutant relative to wild-type plants. *, P<0.05; ** P<0.005; ***, P<0.001.

1. A transgenic plant or plant part comprising a chimeric gene integrated in its genome, characterized in that said chimeric gene comprises a transcription regulatory sequence active in plant cells operably linked to a nucleic acid sequence selected from the group consisting of:
(a) a nucleic acid sequence encoding a protein of SEQ ID NO: 1;
(b) a nucleic acid sequence encoding a protein having at least 70% amino acid identity to SEQ ID NO: 1 over the entire length;
(c) a sense and/or antisense fragment of the sequence of (a) or (b),
wherein said plant or plant part is modified in its growth during exposure to one or more biotic and/or abiotic stresses compared to a non-transgenic plant or plant part.

2. The plant or plant part according to claim 1, wherein said growth is arrested in a reversible manner, such that normal growth resumes upon elimination of said stress.

3. The plant according to claim 1 or 2, wherein the growth of the primary inflorescence, the stem and/or axillary shoots or the sprouting of underground storage organs or embryo growth during seed maturation or dormancy is modified.

4. The transgenic plant according to claims 1 to 3, wherein said biotic and/or abiotic stress is selected from the group consisting of: cold stress, heat stress, salinity, wind, drought stress, water deficiency, water logging, metal stress, nitrogen stress, pest or pathogen damage.

5. The plant according to any one of the preceding claims, wherein said transcription regulatory sequence is selected from the group consisting of: a constitutive promoter, a inducible promoter, a tissue-specific promoter and a developmentally regulated promoter.

6. The plant according to any one of the preceding claims, wherein the plant is selected from a genus of the group consisting of: Zea, Oryza, Triticum, Lycopersicon, Solanum, Hordeum, Brassica, Glycine, Phaseolus, Avena, Sorghum, Gossypium, Beta, Lactuca, Daucus, Apium, Ipomoea Manihot, Colocasia, Raphanus, Dioscorea, Helianthus and Stachys.

7. A seed, underground storage organ, fruit, leaf or flower of a plant according to any one of the preceding claims and comprising the chimeric gene.

8. A underground storage organ according to claim 6, wherein said underground storage organ is a potato tuber.

9. A chimeric gene comprising a promoter active in plant cells, operably linked to a nucleic acid sequence a nucleic acid sequence selected from the group consisting of:
(a) a nucleic acid sequence encoding a protein of SEQ ID NO: 1;
(b) a nucleic acid sequence encoding a protein having at least 98% amino acid identity to SEQ ID NO: 1 over the entire length;
(c) a sense and/or antisense fragment of the sequence of (a) or (b).

10. A vector comprising the chimeric gene according to claim 9.

11. Use of a nucleic acid sequence encoding a chromatin remodeling protein for the generation of transgenic plants or plant parts having modified growth characteristics, characterized in that the nucleic acid sequence is selected from the group consisting of:
(a) a nucleic acid sequence encoding a protein of SEQ ID NO: 1;
(b) a nucleic acid sequence encoding a protein having at least 70% amino acid identity to SEQ ID NO: 1 over the entire length;
(c) a fragment of at least 15 consecutive nucleotides of the sequence of (a) or (b).

12. Use according to claim 13, wherein the modified growth characteristics are one or more of the group consisting of:
(a) biotic and/or abiotic stress-dependent growth arrest or retardation;
(b) dormancy-like growth arrest of underground storage organs;
(c) delayed or suppressed bolting of leaf vegetables; and
(d) embryo growth arrest during seed maturation or seed dormancy.

13. A method for identifying genes involved in plant growth retardation, growth arrest or dormancy comprising the steps of:
(a) generating a transgenic plant or plant part which express the protein of SEQ ID NO: 1 or a protein comprising at least 70% amino acid identity to SEQ ID NO: 1 over the entire length and which is modified in its growth during exposure to one or more biotic and/or abiotic stresses compared to a non-transgenic plant or plant part; and
(b) identifying genes or gene transcripts which are differentially expressed in one or more tissues of the transgenic plants of (a) compared to non-transgenic controls.
